(19) United States
(12) Patent Application Publication
(10) Pub. No.: US 2003/0008848 A1
(43) Pub. Date: Jan. 9, 2003

(54) Small molecules useful in the treatment of inflammatory disease

(76) Inventors: Roman Wolfgang Fleck, Greenwich, CT (US); Terence Alfred Kelly, Ridgefield, CT (US); Jin Mi Kim, Danbury, CT (US); Jinbo Lee, Southbury, CT (US); Rene Marc Lemieux, Plantsville, CT (US); Ronald John Sorcek, Bethel, CT (US); Jiang-Ping Wu, Danbury, CT (US)

Correspondence Address: BOEHRINGER INGELHEIM CORPORATION, 900 RIDGEBURY ROAD, P O BOX 368, RIDGEFIELD, CT, 06877, US

(21) Appl. No.: 10/011,070

(22) Filed: Dec. 5, 2001

Related U.S. Application Data
(60) Provisional application No. 60/256,811, filed on Dec. 19, 2000.

(51) Int. Cl. C 07D 4 3/14; C 07D 4 1/14; A61K 31/675

(52) U.S. Cl. 514/90; 514/241; 514/248; 514/252.03; 514/252.06; 514/255.05; 514/263.2; 514/314; 514/333; 514/338; 514/359; 514/393; 514/403; 514/412; 544/195; 544/212; 544/232; 544/235; 544/238; 544/244; 544/277; 544/405; 546/273.1; 546/256

(57) ABSTRACT

Compound of the formula I, II, III or IV

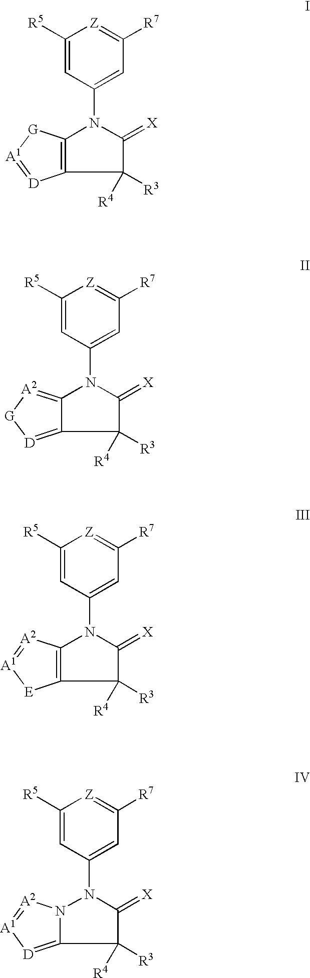

which are useful for treating or preventing inflammatory and immune cell-mediated diseases.

### RELATED APPLICATIONS

Benefit of Provisional Application Serial No. 60/256,81 1, filed Dec. 19, 2000, is hereby claimed.

### FIELD OF THE INVENTION

The present invention relates generally to a series of novel small molecules, their synthesis and their use in the treatment of inflammatory disease.

### BACKGROUND OF THE INVENTION

Research spanning the last decade has helped to elucidate the molecular events attending cell-cell interactions in the body, especially those events involved in the movement and activation of cells in the immune system. See generally, Springer, T. Nature, 1990, 346, 425-434. Cell surface proteins, and especially the Cellular Adhesion Molecules (“CAMs”) and “Leukointegrins”, including LFA-1, MAC-1 and gp150.95 (referred to in WHO nomenclature as CD18/CD11a, CD18/CD11b, and CD18/CD11 c, respectively) have correspondingly been the subject of pharmaceutical research and development having as its goal the intervention in the processes of leukocyte extravasation to sites of injury and leukocyte movement to distinct targets. For example, it is presently believed that prior to the leukocyte extravasation, which is a mandatory component of the inflammatory response, activation of integrins constitutively expressed on leukocytes occurs and is followed by a tight ligand/receptor interaction between integrins (e.g., LFA-1) and one or several distinct intercellular adhesion molecules (ICAMs) designated ICAM-1, ICAM-2, ICAM-3 or ICAM-4 which are expressed on blood vessel endothelial cell surfaces and on other leukocytes. The interaction of the CAMs with the Leukointegrins is a vital step in the normal functioning of the immune system. Immune processes such as antigen presentation, T-cell mediated cytotoxicity and leukocyte extravasation all require cellular adhesion mediated by ICAMs interacting with the Leukointegrins. See generally Kishimoto, T. K.; Rothlein; R. R. Adv. Pharmacol. 1994, 25, 117-138 and Diamond, M.; Springer, T. Current Biology, 1994, 4, 506-532.

A group of individuals has been identified which lack the appropriate expression of Leukointegrins, a condition termed “Leukocyte Adhesion Deficiency” (Anderson, D. C.; et al., Fed. Proc. 1985, 44, 2671-2677 and Anderson, D. C.; et al., J. Infect. Dis. 1985, 152, 668-689). These individuals are unable to mount a normal inflammatory and/or immune response(s) due to an inability of their cells to adhere to cellular substrates. These data show that immune reactions are mitigated when lymphocytes are unable to adhere in a normal fashion due to the lack of functional adhesion molecules of the CD18 family. By virtue of the fact that LAD patients who lack CD18 cannot mount an inflammatory response, it is believed that antagonism of CD18, CD11/ICAM interactions will also inhibit an inflammatory response.

It has been demonstrated that the antagonism of the interaction between the CAMs and the Leukointegrins can be realized by agents directed against either component. Specifically, blocking of the CAMs, such as for example ICAM-1, or the Leukointegrins, such as for example LFA-1, by antibodies directed against either or both of these molecules effectively inhibits inflammatory responses. In vitro models of inflammation and immune response inhibited by antibodies to CAMs or Leukointegrins include antigen or mitogen-induced lymphocyte proliferation, homotypic aggregation of lymphocytes, T-cell mediated cytolysis and antigen-specific induced tolerance. The relevance of the in vitro studies are supported by in vivo studies with antibodies directed against ICAM-1 or LFA-1. For example, antibodies directed against LFA-1 can prevent thyroid graft rejection and prolong heart allograft survival in mice (Gorski, A.; Immunology Today, 1994, 15, 251-255). Of greater significance, antibodies directed against ICAM-1 have shown efficacy in vivo as anti-inflammatory agents in human diseases such as renal allograft rejection and rheumatoid arthritis (Rothlein, R. R.; Scharschmidt, L., in: Adhesion Molecules; Wegner, C. D., Ed.; 1994, 1-38, Cosimi, C. B.; et al., J. Immunol. 1990, 144, 4604-4612 and Kavanaugh, A.; et al., Arthritis Rheum. 1994, 37, 992-1004) and antibodies directed against LFA-1 have demonstrated immunosuppressive effects in bone marrow transplantation and in the prevention of early rejection of renal allografts (Fischer, A.; et al., Lancet, 1989, 2, 1058-1060 and Le Mauff, B.; et al., Transplantation, 1991, 52, 291-295).

It has also been demonstrated that a recombinant soluble form of ICAM-1 can act as an inhibitor of the ICAM-1 interaction with LFA-1 . Soluble ICAM-1 acts as a direct antagonist of CD18,CD11/ICAM-1 interactions on cells and shows inhibitory activity in in vitro models of immune response such as the human mixed lymphocyte response, cytotoxic T cell responses and T cell proliferation from diabetic patients in response to islet cells (Becker, J. C.; et al., J. Immunol. 1993, 151, 7224 and Roep, B. O.; et al., Lancet, 1994, 343, 1590).

Thus, the prior art has demonstrated that large protein molecules which antagonize the binding of the CAMs to the Leukointegrins have therapeutic potential in mitigating inflammatory and immunological responses often associated with the pathogenesis of many autoimmune or inflammatory diseases. However proteins have significant deficiencies as therapeutic agents, including the inability to be delivered orally and potential immunoreactivity which limits the utility of theses molecules for chronic administration. Furthermore, protein-based therapeutics are generally expensive to produce.

Several small molecules have been described in the literature which affect the interaction of CAMs and Leukointegrins. A natural product isolated from the root of Trichilia rubra was found to be inhibitory in an in vitro cell binding assay (Musza, L. L.; et al, Tetrahedron, 1994, 50, 11369-11378). One series of molecules (Boschelli, D. H.; et al., J. Med. Chem. 1994, 37, 717 and Boschelli, D. H.; et al., J. Med. Chem. 1995, 38, 4597-4614) was found to be orally active in a reverse passive Arthus reaction, an induced model of inflammation that is characterized by neutrophil accumulation (Chang, Y. H.; et al., Eur. J. Pharmacol. 1992, 69, 155-164). Another series of molecules was also found to be orally active in a delayed type hypersensitivity reaction in rats (Sanfilippo, P. J.; et al., J. Med. Chem. 1995, 38, 1057-1059). All of these molecules appear to act nonspecifically, either by inhibiting the transcription of ICAM-1 along with other proteins or act intracellularly to inhibit the activation of the Leukointegrins by an unknown mechanism. None of the molecules directly antagonize the interaction of the CAMs with the Leukointegrins. Due to lack of potency, lack of selectivity and lack of a specific mechanism of action, the described small molecules are not likely to be satisfactory for therapeutic use.

It follows that small molecules having the similar ability as large protein molecules to directly and selectively antagonize the binding of the CAMs to the Leukointegrins would make preferable therapeutic agents. WO9839303 discloses a class of small molecule inhibitors of the interaction of LFA-1 and ICAM-1. WO9911258 discloses that the fungal metabolite mevinolin and derivatives bind to LFA-1 and disrupt the interaction of LFA-1 and ICAM-1. WO9949856 discloses a class of peptidomimetic inhibitors of ICAM binding to LFA-1 and Mac-1. WO0039081, WO0059880 and WO0059878 all disclose small molecule aryl thioethers as inhibitors of the interaction of LFA-1 and ICAM-1. WO0107440 discloses small molecule imidazoimidazoles and triazoles as inhibitors of the interaction of LFA-1 and ICAM-1.

### SUMMARY OF THE INVENTION

A first aspect of the invention comprises a method for treating or preventing inflammatory and immune cell-mediated diseases by the administration of certain novel small molecules. These compounds act by inhibiting the interaction of cellular adhesion molecules, specifically by antagonizing the binding of human intercellular adhesion molecules (including ICAM-1, ICAM-2 and ICAM-3) to the Leukointegrins (especially CD18/CD11a). A second aspect of the invention comprises novel small molecules having the above-noted therapeutic activities. A third aspect of the invention comprises methods for making these novel compounds. A final aspect of the invention comprises pharmaceutical compositions comprising the above-mentioned compounds suitable for the prevention or treatment of inflammatory and immune cell-mediated conditions.

### DETAILED DESCRIPTION OF THE INVENTION

In its broadest aspect, the invention comprises compounds of the formulas I, II, III and IV

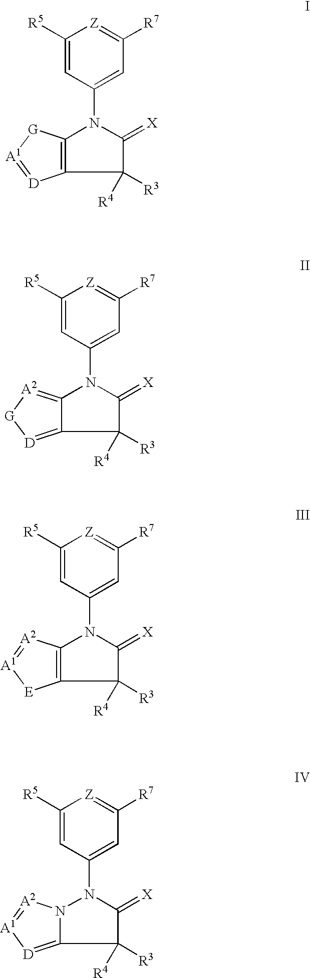

wherein:

A¹ is ═N—, ═C(H)—, or ═C(R′)— wherein R′ is halogen, —CN, —Oalkyl, —CO₂alkyl or —SO₂alkyl, wherein the foregoing alkyl moieties are of 1 to 3 carbon atoms;

A² is ═N— or ═C(H)—;

D is ═N—, ═C(R¹)—, ═C(H)—, ═C(SO₂R¹)—, ═C(S(O)R¹)—, ═C(C(O)R¹)—, ═C(C(O)H)—, ═C(SR^{1a})—, ═C(OR^{1a})— or ═C(NHR^{1a})—,

wherein R¹ is selected from the class consisting of:

(A) —R¹⁰⁰, which is:

branched or unbranched alkyl of 1 to 6 carbon atoms, alkenyl of 2 to 6 carbon atoms or cycloalkyl or cycloalkenyl of 3 to 6 carbon atoms, in which alkyl, alkenyl, cycloalkyl or cycloalkenyl group one or more hydrogen atoms are optionally and independently replaced with:

(i) halogen,

(ii) oxo,

(iii) aryl or heteroaryl which is selected from the class consisting of phenyl, naphthyl, indolyl, thiophenyl, pyridyl, pyrimidinyl, furyl, pyrrolyl, oxazolyl, thiazolyl, pyrazolyl, isoxazolyl, imidazolyl, isothiazolyl, oxadiazolyl, triazolyl, thiadiazolyl, pyridazinyl, pyrazinyl, triazinyl, indolyzinyl, isoindolyl, benzo[b]furanyl, benzo[b]thiophenyl, indazolyl, benzthiazolyl, benzimidazolyl, quinolinyl, isoquinolinyl, purinyl, quinolizinyl, cinnolinyl, pthalaninyl, quinoxalinyl, napthyridinyl, pteridinyl and quinazolinyl, wherein one or more hydrogen atoms of said aryl or heteroaryl group are optionally and independently replaced with:

(a) alkyl of 1 to 3 carbon atoms,

(b) —COOH,

(c) —SO₂OH,

(d) —PO(OH)₂,

(e) a group of the formula —COOR⁸, wherein R⁸ is straight or branched alkyl of 1 to 5 carbon atoms or cycloalkyl of 3 to 5 carbon atoms,

(f) a group of the formula —NR⁹R¹⁰, wherein R⁹ and R¹⁰ are each independently a hydrogen atom, alkyl of 1 to 6 carbon atoms, cycloalkyl of 3 to 6 carbon atoms or acyl of 1 to 7 carbon atoms, or wherein R⁹ and R¹⁰ constitute a saturated hydrocarbon bridge of 3 to 5 carbon atoms which together with the nitrogen atom between them form a heterocyclic ring,

(g) a group of the formula —CONR¹¹R¹², wherein R¹¹ and R¹² are each independently a hydrogen atom, alkyl of 1 to 6 carbon atoms or cycloalkyl of 3 to 6 carbon atoms, or wherein R¹¹ and R¹² constitute a saturated hydrocarbon bridge of 3 to 5 carbon atoms which together with the nitrogen atom between them form a heterocyclic ring, and wherein one carbon atom in said hydrocarbon bridge is optionally replaced by —O—, —S—, S(O)—, SO₂—, —NH—, or —NMe—,

(h) a group of the formula —OR¹³, wherein R¹³ is a hydrogen atom, or an alkyl or acyl group of 1 to 7 carbon atoms,

(i) a group of the formula —SR¹⁴, wherein R¹⁴ is a hydrogen atom, or an alkyl or acyl group of 1 to 7 carbon atoms,

(j) —CN, or

(k) an amidino group of the formula

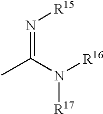

wherein R¹⁵, R¹⁶ and R¹⁷ are each, independently, a hydrogen atom or alkyl of 1 to 3 carbon atoms and wherein two of R¹⁵, R¹⁶ and R¹⁷ may additionally constitute a saturated hydrocarbon bridge of 3 to 5 carbon atoms which together with the nitrogen atom(s) between them form a heterocyclic ring,

(l) halogen,

(m) a group of the formula —NHCONHalkyl, wherein the alkyl moiety contains 1 to 3 carbon atoms,

(n) a group of the formula —NHCOOalkyl, wherein the alkyl moiety contains 1 to 3 carbon atoms,

(iv) a group of the formula —COOR¹⁸, wherein R¹⁸ is straight or branched alkyl of 1 to 7 carbon atoms or cycloalkyl of 3 to 6 carbon atoms,

(v) —CN,

(vi) a group of the formula —CONR¹⁹R²⁰, wherein R¹⁹ and R²⁰ are each, independently, a hydrogen atom, alkyl of 1 to 6 carbon atoms or cycloalkyl of 3 to 6 carbon atoms, or wherein R¹⁹ and R²⁰ constitute a saturated hydrocarbon bridge of 3 to 5 carbon atoms which together with the nitrogen atom between them form a heterocyclic ring, and wherein one carbon atom in said hydrocarbon bridge is optionally replaced by —O—, —S—, S(O)—, SO₂—, —NH—, or —NMe—,

(vii) a group of the formula —OR²¹, wherein R²¹ is a hydrogen atom, or a straight or branched alkyl or acyl group of 1 to 7 carbon atoms, wherein one or more hydrogen atoms of said alkyl or acyl group are optionally replaced with a group independently selected from the class consisting of —OH, —Oalkyl (wherein the alkyl moiety contains 1 to 6 carbon atoms), —NH₂, —NHMe and —NMe₂,

(viii) a group of the formula —SR²², wherein R²² is a hydrogen atom, or an alkyl or acyl group of 1 to 7 carbon atoms, wherein one or more hydrogen atoms of said alkyl or acyl group are optionally replaced with a group independently selected from the class consisting of —OH, —Oalkyl (wherein the alkyl moiety is 1 to 6 carbon atoms), —NH₂, —NHMe and —NMe₂,

(ix) a group of the formula —NR²³R²⁴, wherein R²³ and R²⁴ are each, independently,

(a) a hydrogen atom,

(b) straight or branched alkyl or acyl of 1 to 7 carbon atoms or cycloalkyl of 3 to 7 carbon atoms, wherein said one or more hydrogen atoms of said alkyl or acyl group are optionally replaced with a group independently selected from the class consisting of —OH, —Oalkyl (wherein the alkyl moiety is 1 to 6 carbon atoms), —NH₂, —NHMe and —NMe₂,

(c) a group of the formula —(CH₂)ₘCOOH, wherein m is 0, 1 or 2,

(d) a group of the formula —(CH₂)ₘCOOR²⁵, wherein n is 0, 1 or 2, and wherein R²⁵ is straight or branched alkyl of 1 to 6 carbon atoms, or

(e) a group of the formula —(CH₂)ₘCONHR²⁵, wherein n is 0, 1 or 2, and wherein R²⁵ is straight or branched alkyl of 1 to 6 carbon atoms,

(x) a quaternary group of the formula

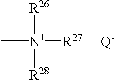

wherein R²⁶, R²⁷ and R²⁸ are each, independently, a branched or unbranched alkyl group of 1 to 7 carbon atoms and Q⁻ is a pharmaceutically acceptable counter ion,

(xi) a saturated, or partially unsaturated heterocyclic group consisting of 3 to 7 ring atoms selected from N, O, C and S, including but not limited to imidazolinyl, imidazolidinyl, pyrrolinyl, pyrrolidinyl, piperidinyl, piperazinyl, morpholinyl, thiomorpholinyl, thiazolidinyl, azepinyl, tetrahydropyranyl, tetrahydrofuranyl, benzodioxolyl, tetrahydrothiophenyl and sulfolanyl, wherein said heterocyclic group is optionally mono- or polysubstituted with oxo, and

(xii) a cycloalkyl group of 3 to 7 carbon atoms,

(B) branched or unbranched carboxylic acid groups of 3 to 6 carbon atoms,

(C) branched or unbranched phosphonic acid groups of 2 to 6 carbon atoms,

(D) branched or unbranched sulfonic acid groups of 2 to 6 carbon atoms,

(E) amidino groups of the formula

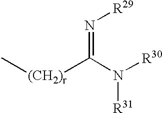

wherein r is 2, 3, 4, 5 or 6, and R²⁹, R³⁰ and R³¹ are each, independently, a hydrogen atom or alkyl of 1 to 3 carbon atoms, and wherein two of R²⁹, R³⁰ and R³¹ may additionally constitute a saturated hydrocarbon bridge of 3 to 5 carbon atoms which together with the nitrogen atom(s) between them form a heterocyclic ring,

(F) guanidino groups of the formula

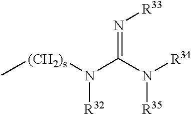

wherein s is 2, 3, 4, 5 or 6, and R³², R³³, R³⁴ and R³⁵ are each, independently, a hydrogen atom or alkyl of 1 to 3 carbon atoms, and wherein two of R³², R³³, R³⁴ and R³⁵ may additionally constitute a saturated hydrocarbon bridge of 3 to 5 carbon atoms which together with the nitrogen atom(s) between them form a heterocyclic ring,

(G) aryl or heteroaryl which is selected from the class consisting of phenyl, naphthyl, indolyl, thiophenyl, pyridyl, pyrimidinyl, furyl, pyrrolyl, oxazolyl, thiazolyl, pyrazolyl, isoxazolyl, imidazolyl, isothiazolyl, oxadiazolyl, triazolyl, thiadiazolyl, pyridazinyl, pyrazinyl, triazinyl, indolyzinyl, isoindolyl, benzo[b]furanyl, benzo[b]thiophenyl, indazolyl, benzthiazolyl, benzimidazolyl, quinolinyl, isoquinolinyl, purinyl, quinolizinyl, cinnolinyl, pthalaninyl, quinoxalinyl, napthyridinyl, pteridinyl and quinazolinyl, wherein one or more hydrogen atoms of said aryl or heteroaryl group are optionally and independently replaced with:

(i) alkyl of 1 to 3 carbon atoms,

(ii) —COOH,

(iii) —SO₂OH,

(iv) —PO(OH)₂,

(v) a group of the formula —COOR³⁶, wherein R³⁶ is straight or branched alkyl of 1 to 5 carbon atoms or cycloalkyl of 3 to 5 carbon atoms,

(vi) a group of the formula —NR³⁷R³⁸, wherein R³⁷ and R³⁸ are each, independently, a hydrogen atom, alkyl of 1 to 6 carbon atoms, cycloalkyl of 3 to 6 carbon atoms or acyl of 1 to 7 carbon atoms, or wherein R³⁷ and R³⁸ constitute a saturated hydrocarbon bridge of 3 to 5 carbon atoms which together with the nitrogen atom between them form a heterocyclic ring,

(vii) a group of the formula —CONR³⁹R⁴⁰, wherein R³⁹ and R⁴⁰ are each, independently, a hydrogen atom, alkyl of 1 to 6 carbon atoms or cycloalkyl of 3 to 6 carbon atoms, or wherein R³⁹ and R⁴⁰ constitute a saturated hydrocarbon bridge of 3 to 5 carbon atoms which together with the nitrogen atom between them form a heterocyclic ring, and wherein one carbon atom in said hydrocarbon bridge is optionally replaced by —O—, —S—, S(O)—, SO₂—, —NH—, or —NMe—,

(viii) a group of the formula —OR⁴¹, wherein R⁴¹ is a hydrogen atom, or an alkyl or acyl group of 1 to 7 carbon atoms,

(ix) a group of the formula —SR⁴², wherein R⁴² is a hydrogen atom, or an alkyl or acyl group of 1 to 7 carbon atoms,

(x) —CN, or

(xi) an amidino group of the formula

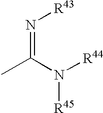

wherein R⁴³, R⁴⁴ and R⁴⁵ are each, independently, a hydrogen atom or alkyl of 1 to 3 carbon atoms, and wherein two of R⁴³, R⁴⁴ and R⁴⁵ may additionally constitute a saturated hydrocarbon bridge of 3 to 5 carbon atoms which together with the nitrogen atom(s) between them form a heterocyclic ring,

(H) groups of the formula —NR⁴⁶R⁴⁷, wherein R⁴⁶ and R⁴⁷ are each independently a hydrogen atom, phenyl which is optionally mono- or polysubstituted with halogen, or R¹⁰⁰, wherein R¹⁰⁰ is as hereinbefore defined,

(I) saturated or unsaturated heterocyclic groups consisting of 3 to 7 ring atoms selected from N, O, C and S, or bicyclic heterocyclic groups consisting of 8 to 11 atoms selected from N, O, C and S, including but not limited to imidazolinyl, imidazolidinyl, pyrrolinyl, pyrrolidinyl, piperidinyl, piperazinyl, morpholinyl, thiomorpholinyl, thiazolidinyl, azepinyl, tetrahydropyranyl, tetrahydrofuranyl, benzodioxolyl, tetrahydrothiophenyl and sulfolanyl, wherein said heterocyclic group is optionally mono- or poly-substituted with moieties selected from the class consisting of:

(i) oxo,

(ii) —OR¹⁰¹, wherein R¹⁰¹ is:

(a) a hydrogen atom,

(b) alkyl of 1 to 7 carbons, wherein any hydrogen atom of said alkyl group is optionally replaced with —OH, —OR¹¹⁰ (wherein R¹¹⁰ is an alkyl moiety of 1 to 6 carbon atoms), —NH₂, —NHMe or —NMe₂,

(c) acyl of 1 to 7 carbons, wherein any hydrogen atom of said acyl group is optionally replaced with —OH, —OR¹¹¹ (wherein R¹¹¹ is an alkyl moiety of 1 to 6 carbon atoms), —NH₂, —NHMe or —NMe₂,

(d) —CONR¹⁰²R¹⁰³, wherein R¹⁰² and R¹⁰³ are each independently a hydrogen atom or alkyl of 1 to 7 atoms, or wherein R¹⁰² and R¹⁰³ constitute a saturated hydrocarbon bridge of 3 to 5 carbon atoms which together with the nitrogen atom between them form a heterocyclic ring, and wherein one carbon atom in said hydrocarbon bridge is optionally replaced by —O—, —S—, S(O)—, SO₂—, —NH—, or —NMe—, or

(e) —COOR¹⁰⁴, wherein R¹⁰⁴ is alkyl of 1 to 7 atoms,

(iii) —CONR¹⁰⁵R¹⁰⁶, wherein R¹⁰⁵ and R¹⁰⁶ are each independently:

(a) a hydrogen atom,

(b) straight or branched alkyl of 1 to 7 atoms or cycloalkyl of 3 to 7 atoms,

(c) benzoyl,

(d) benzyl or

(e) phenyl, wherein said phenyl ring is optionally mono- or polysubstituted with —OR¹¹²,wherein R¹¹² is alkyl of 1 to 6 carbon atoms,

or, wherein R¹⁰⁵ and R¹⁰⁶ constitute a saturated hydrocarbon bridge of 3 to 5 carbon atoms which together with the nitrogen atom between them form a heterocyclic ring, and wherein one carbon atom in said hydrocarbon bridge is optionally replaced by —O—, —S—, S(O)—, SO₂—, —NH—, or —NMe—,

(iv) —COOR¹⁰⁷, wherein R¹⁰⁷ is a hydrogen atom, or straight or branched alkyl of 1 to 7 carbon atoms,

(v) straight or branched alkyl of 1 to 7 carbon atoms, alkenyl or alkynyl of 2 to 7 carbon atoms, or cycloalkyl of 3 to 7 carbons, wherein one or more hydrogen atoms of said alkyl, alkenyl, alkynyl or cycloalkyl group is optionally replaced with a moiety independently selected from the class consisting of:

(a) oxo,

(b) —OH,

(c) —OR¹¹³, wherein R¹¹³ is alkyl of 1 to 6 carbon atoms,

(d) —OCOCH₃,

(e) —NH₂,

(f) —NHMe,

(g) —NMe₂,

(h) —CO₂H, and

(i) —CO₂ R¹¹⁴ wherein R¹¹⁴ is alkyl of 1 to 3 carbon atoms, or cycloalkyl of 3 to 7 carbons,

(vi) acyl of 1 to 7 carbon atoms, which may be straight, branched or cyclic, and wherein one or more hydrogen atoms of said acyl group is optionally replaced with a moiety independently selected from the class consisting of:

(a) —OH,

(b) —OR¹¹⁵, wherein R¹¹⁵ is alkyl of 1 to 6 carbon atoms,

(c) —NH₂,

(d) —NHMe,

(e) —NMe₂,

(f) —NHCOMe,

(g) oxo,

(h) —CO₂R¹¹⁶, wherein R¹¹⁶ is alkyl of 1 to 3 carbon atoms,

(i) —CN,

(j) the halogen atoms,

(k) heterocycles selected from the class consisting of imidazolinyl, imidazolidinyl, pyrrolinyl, pyrrolidinyl, piperidinyl, piperazinyl, morpholinyl, thiomorpholinyl, thiazolidinyl, azepinyl, tetrahydropyranyl, tetrahydrofuranyl, benzodioxolyl, tetrahydrothiophenyl and sulfolanyl, and

(l) aryl or heteroaryl selected from the class consisting of phenyl, naphthyl, indolyl, thiophenyl, pyridyl, pyrimidinyl, furyl, pyrrolyl, oxazolyl, thiazolyl, pyrazolyl, isoxazolyl, imidazolyl, isothiazolyl, oxadiazolyl, triazolyl, thiadiazolyl, pyridazinyl, pyrazinyl, triazinyl, indolyzinyl, isoindolyl, benzo[b]furanyl, benzo[b]thiophenyl, indazolyl, benzthiazolyl, benzimidazolyl, quinolinyl, isoquinolinyl, purinyl, quinolizinyl, cinnolinyl, pthalaninyl, quinoxalinyl, napthyridinyl, pteridinyl and quinazolinyl,

(vii) —SO₂R¹⁰⁸, wherein R¹⁰⁸ is:

(a) aryl or heteroaryl which is selected from the group consisting of phenyl, naphthyl, indolyl, thiophenyl, pyridyl, pyrimidinyl, furyl, pyrrolyl, oxazolyl, thiazolyl, pyrazolyl, isoxazolyl, imidazolyl, isothiazolyl, oxadiazolyl, triazolyl, thiadiazolyl, pyridazinyl, pyrazinyl, triazinyl, indolyzinyl, isoindolyl, benzo[b]furanyl, benzo[b]thiophenyl, indazolyl, benzthiazolyl, benzimidazolyl, quinolinyl, isoquinolinyl, purinyl, quinolizinyl, cinnolinyl, pthalaninyl, quinoxalinyl, napthyridinyl, pteridinyl and quinazolinyl, wherein said aryl or heteroaryl moiety is optionally substituted with one or more moieties selected from the class consisting of the halogen atoms, straight or branched alkyl of 1 to 6 carbons, and —OR¹¹⁷ (wherein R¹¹⁷ is hydrogen or alkyl of 1 to 6 carbon atoms),

(b) a heterocyclic group selected from the class consisting of imidazolinyl, imidazolidinyl, pyrrolinyl, pyrrolidinyl, piperidinyl, piperazinyl, morpholinyl, thiomorpholinyl, thiazolidinyl, azepinyl, tetrahydropyranyl, tetrahydrofuranyl, benzodioxolyl, tetrahydrothiophenyl and sulfolanyl, wherein said heterocyclic group is optionally substituted with one or more moieties selected from the class consisting of the halogen atoms, straight or branched alkyl of 1 to 6 carbons, and —OR¹¹⁸ (wherein R¹¹⁸ is hydrogen or alkyl of 1 to 6 carbon atoms), or

(c) straight or branched alkyl of 1 to 7 atoms, wherein said alkyl moiety is optionally substituted with one or more moieties selected from the class consisting of the halogen atoms, straight or branched alkyl of 1 to 6 carbons, and —OR¹¹⁹ (wherein R¹¹⁹ is hydrogen or alkyl of 1 to 6 carbon atoms),

(viii) —COR¹⁰⁹, wherein R¹⁰⁹ is:

(a) aryl or heteroaryl which is selected from the class consisting of phenyl, naphthyl, indolyl, thiophenyl, pyridyl, pyrimidinyl, furyl, pyrrolyl, oxazolyl, thiazolyl, pyrazolyl, isoxazolyl, imidazolyl, isothiazolyl, oxadiazolyl, triazolyl, thiadiazolyl, pyridazinyl, pyrazinyl, triazinyl, indolyzinyl, isoindolyl, benzo[b]furanyl, benzo[b]thiophenyl, indazolyl, benzthiazolyl, benzimidazolyl, quinolinyl, isoquinolinyl, purinyl, quinolizinyl, cinnolinyl, pthalaninyl, quinoxalinyl, napthyridinyl, pteridinyl and quinazolinyl, wherein said aryl or heteroaryl moiety is optionally substituted with one or more moieties selected from the class consisting of the halogen atoms, straight or branched alkyl of 1 to 6 carbons, and —OR¹²⁰ (wherein R¹²⁰ is hydrogen or alkyl of 1 to 6 carbon atoms),

(b) a heterocyclic group selected from the class consisting of imidazolinyl, imidazolidinyl, pyrrolinyl, pyrrolidinyl, piperidinyl, piperazinyl, morpholinyl, thiomorpholinyl, thiazolidinyl, azepinyl, tetrahydropyranyl, tetrahydrofuranyl, benzodioxolyl, tetrahydrothiophenyl and sulfolanyl, wherein said heterocyclyl is optionally substituted with one or more halogen, straight or branched alkyl of 1 to 6 carbons, or —OR¹²¹ (wherein R¹²¹ is hydrogen or alkyl of 1 to 6 carbon atoms), or

(c) straight or branched alkyl of 1 to 7 atoms, wherein said alkyl moiety is optionally substituted with one or more moieties selected from the class consisting of the halogen atoms, straight or branched alkyl of 1 to 6 carbons, and —OR¹²² (wherein R¹²² is hydrogen or alkyl of 1 to 6 carbon atoms),

(ix) —CHO,

(x) the halogen atoms, and

(xi) aryl or heteroaryl which is selected from the class consisting of phenyl, naphthyl, indolyl, thiophenyl, pyridyl, pyrimidinyl, furyl, pyrrolyl, oxazolyl, thiazolyl, pyrazolyl, isoxazolyl, imidazolyl, isothiazolyl, oxadiazolyl, triazolyl, thiadiazolyl, pyridazinyl, pyrazinyl, triazinyl, indolyzinyl, isoindolyl, benzo[b]furanyl, benzo[b]thiophenyl, indazolyl, benzthiazolyl, benzimidazolyl, quinolinyl, isoquinolinyl, purinyl, quinolizinyl, cinnolinyl, pthalaninyl, quinoxalinyl, napthyridinyl, pteridinyl and quinazolinyl,

(J) the halogen atoms, and

(K) —CN and,

wherein R^{1a} is R¹⁰⁰;

E is —N(R¹)—, —N(H)—, —N(SO₂R¹)—, —N(S(O)R¹)— or —N(C(O)R¹)—, where R¹ is as defined above;

G is —O—, —S— or —N(H)—;

X is an oxygen or sulfur atom;

R³ is:

(A) a hydrogen atom, or

(B) branched or unbranched alkyl of 1 to 3 carbon atoms or cycloalkyl of 3 to 5 carbon atoms wherein said alkyl or cycloalkyl group is optionally substituted with:

(i) a group of the formula —OR⁴⁸, wherein R⁴⁸ is a hydrogen atom, or an alkyl or acyl group of 1 to 7 carbon atoms, or

(ii) a group of the formula —NR⁴⁹R⁵⁰, wherein R⁴⁹ and R⁵⁰ are each, independently, a hydrogen atom, alkyl of 1 to 2 carbon atoms, or acyl of 1 to 2 carbon atoms;

R⁴ is a group of the formula —(CR⁵¹R⁵²)ₓ(CR⁵³R⁵⁴)_{y}R⁵⁵, wherein,

x is 0 or 1,

y is 0 or 1,

R⁵¹, R⁵² and R⁵³ are each, independently:

(A) a hydrogen atom,

(B) a group of the formula —OR⁵⁶, wherein R⁵⁶ is a hydrogen atom, or an alkyl or acyl group of 1 to 7 carbon atoms, or

(C) branched or unbranched alkyl of 1 to 3 carbon atoms or cycloalkyl of 3 to 5 carbon atoms,

R⁵⁴ is:

(A) a group of the formula R⁵⁷, wherein R⁵⁷ is independently selected from the same class as is R¹, or

(B) a group of the formula —OR⁵⁸, wherein R⁵⁸ is independently selected from the same class as is R¹;

R⁵⁵ is:

aryl or heteroaryl which is selected from the class consisting of phenyl, naphthyl, indolyl, thiophenyl, pyridyl, pyrimidinyl, furyl, pyrrolyl, oxazolyl, thiazolyl, pyrazolyl, isoxazolyl, imidazolyl, isothiazolyl, oxadiazolyl, triazolyl, thiadiazolyl, pyridazinyl, pyrazinyl, triazinyl, indolyzinyl, isoindolyl, benzo[b]furanyl, benzo[b]thiophenyl, indazolyl, benzthiazolyl, benzimidazolyl, quinolinyl, isoquinolinyl, purinyl, quinolizinyl, cinnolinyl, pthalaninyl, quinoxalinyl, napthyridinyl, pteridinyl and quinazolinyl, wherein one or more of the hydrogen atoms of said aryl or heteroaryl group is optionally and independently replaced with:

(A) R⁵⁹, which is aryl or heteroaryl selected from the class consisting of phenyl, naphthyl, indolyl, thiophenyl, pyridyl, pyrimidinyl, furyl, pyrrolyl, oxazolyl, thiazolyl, pyrazolyl, isoxazolyl, imidazolyl, isothiazolyl, oxadiazolyl, triazolyl, thiadiazolyl, pyridazinyl, pyrazinyl, triazinyl, indolyzinyl, isoindolyl, benzo[b]furanyl, benzo[b]thiophenyl, indazolyl, benzthiazolyl, benzimidazolyl, quinolinyl, isoquinolinyl, purinyl, quinolizinyl, cinnolinyl, pthalaninyl, quinoxalinyl, napthyridinyl, pteridinyl and quinazolinyl, wherein one or more of the hydrogen atoms of said aryl or heteroaryl group is optionally and independently replaced with:

(i) branched or unbranched alkyl of 1 to 6 carbon atoms or cycloalkyl of 3 to 6 carbon atoms, which alkyl or cycloakyl group is optionally mono- or polysubstituted with halogen or oxo,

(ii) a group of the formula —COOR⁶⁰, wherein R⁶⁰ is straight or branched alkyl of 1 to 5 carbon atoms or cycloalkyl of 3 to 5 carbon atoms,

(iii) a group of the formula —NR⁶¹R⁶², wherein R⁶¹ and R⁶² are each, independently, a hydrogen atom, alkyl or fluoroalkyl of 1 to 6 carbon atoms, cycloalkyl of 3 to 6 carbon atoms or acyl of 1 to 7 carbon atoms, or wherein R⁶¹ and R⁶² constitute a saturated hydrocarbon bridge of 3 to 5 carbon atoms which together with the nitrogen atom between them form a heterocyclic ring,

(iv) a group of the formula —CONR⁶³R⁶⁴, wherein R⁶³ and R⁶⁴ are each, independently, a hydrogen atom, alkyl or fluoroalkyl of 1 to 6 carbon atoms or cycloalkyl of 3 to 6 carbon atoms, or wherein R⁶³ and R⁶⁴ constitute a saturated hydrocarbon bridge of 3 to 5 carbon atoms which together with the nitrogen atom between them form a heterocyclic ring,

(v) a group of the formula —OR⁶⁵, wherein R⁶⁵ is a hydrogen atom, or an alkyl, fluoroalkyl or acyl group of 1 to 7 carbon atoms,

(vi) a group of the formula —SR⁶⁶, wherein R⁶⁶ is a hydrogen atom, or an alkyl, fluoroalkyl or acyl group of 1 to 7 carbon atoms,

(vii) —CN,

(viii) nitro, or

(ix) halogen,

(B) methyl, which is optionally mono- or polysubstituted with fluorine atoms and additionally is optionally monosubstituted with R⁵⁹,

(C) branched or unbranched alkyl of 2 to 6 carbon atoms or cycloalkyl of 3 to 6 carbon atoms, which alkyl or cycloakyl group is optionally mono- or polysubstituted with halogen or oxo,

(D) a group of the formula —COOR⁶⁷, wherein R⁶⁷ is straight or branched alkyl of 1 to 5 carbon atoms or cycloalkyl of 3 to 5 carbon atoms,

(E) a group of the formula —NR⁶⁸R⁶⁹, wherein R⁶⁸ and R⁶⁹ are each, independently, a hydrogen atom, alkyl or fluoroalkyl of 1 to 6 carbon atoms, cycloalkyl of 3 to 6 carbon atoms or acyl of 1 to 7 carbon atoms, or wherein R⁶⁸ and R⁶⁹ constitute a saturated hydrocarbon bridge of 3 to 5 carbon atoms which together with the nitrogen atom between them form a heterocyclic ring, and wherein one of R⁶⁸ and R⁶⁹ may additionally be the group R⁵⁹,

(F) a group of the formula —CONR⁷⁰R⁷¹, wherein R⁷⁰ and R⁷¹ are each, independently, a hydrogen atom, alkyl or fluoroalkyl of 1 to 6 carbon atoms or cycloalkyl of 3 to 6 carbon atoms, or wherein R⁷⁰ and R⁷¹ constitute a saturated hydrocarbon bridge of 3 to 5 carbon atoms which together with the nitrogen atom between them form a heterocyclic ring, and wherein one of R⁷⁰ and R⁷¹ may additionally be the group R⁵⁹,

(G) a group of the formula —COR⁷², wherein R⁷² is a hydrogen atom, straight or branched alkyl of 1 to 5 carbon atoms, cycloalkyl of 3 to 5 carbon atoms or R⁵⁹,

(H) a group of the formula —OR⁷³, wherein R⁷³ is a hydrogen atom, an alkyl, fluoroalkyl or acyl group of 1 to 7 carbon atoms, or R⁵⁹,

(I) a group of the formula —SR⁷⁴, wherein R⁷⁴ is a hydrogen atom, an alkyl, fluoroalkyl or acyl group of 1 to 7 carbon atoms, or R⁵⁹,

(J) —CN,

(K) nitro, or

(L) halogen;

R⁵ is Cl or trifluoromethyl;

Z is ═N— or ═C(R⁶)— wherein R⁶ is a hydrogen, fluorine, chlorine, bromine or iodine atom, methyl or trifluoromethyl; and,

R⁷ is a hydrogen, fluorine, chlorine, bromine or iodine atom, methyl, —CN, nitro or trifluoromethyl, with the condition that when Z is ═N— or ═C(H)—, R⁷ is chlorine, trifluoromethyl, —CN or nitro; and pharmaceutically acceptable salts thereof.

It will be appreciated that the compounds of the formulas I, II, III and IV have at least one chiral center. Preferred compounds are those with the absolute stereochemistry depicted below in formulas Ia, Ia, IIIa and IVa.

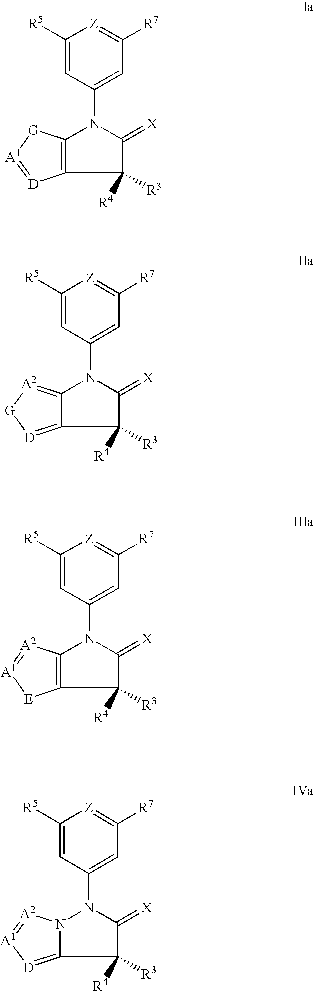

As the term is used herein, a “pharmaceutically acceptable counter ion” is any counter ion generally regarded by those skilled in the pharmaceutical art as being pharmaceutically acceptable. For a discussion of what are pharmaceutically acceptable counter ions, reference may be had to Stephen M. Bergle, Lyle D. Bighley and Donald C. Monkhouse, “Pharmaceutical Salts”, Journal of Pharmaceutical Sciences, 66 (1977), 1-19. By way of non-limiting example, the chloride, bromide, acetate, and sulphate ions are pharmaceutically acceptable counter ions.

Synthesis of the Compounds of the Invention

Compounds of the invention may be prepared by the general methods described below. Typically, reaction progress may be monitored by thin layer chromatography (TLC) if desired. If desired, intermediates and products may be purified by chromatography on silica gel and/or recrystallization, and characterized by one or more of the following techniques: NMR, mass spectroscopy and melting point. Starting materials and reagents are either commercially available or may be prepared by one skilled in the art using methods described in the chemical literature.

The methods described below describe the preparation of intermediates as well as compounds of formulas I, II, III and IV. Intermediates may be further reacted by methods known in the art to provide desired compound of formula I, II, III or IV. Some of these methods are described in U.S. Ser. No. 09/604,312, filed Jun. 27, 2000 and are incorporated herein by reference.

Intermediates used in the preparation of the compounds of formula I may be prepared by the method described below and is outlined in Scheme I.

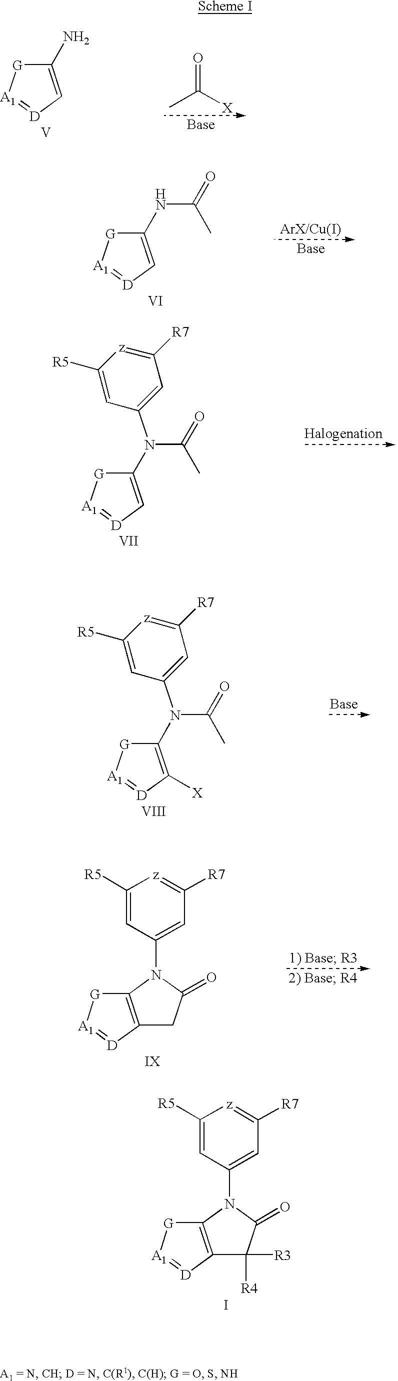

An appropriate amino heterocycle V is treated with an acetylating agent, such as acetyl chloride, in the presence of a base, such as diisopropylethylamine to generate intermediate VI. Compound VI can then be treated with an appropriate aryl halide, in the presence of a copper reagent (Sugahara, S.; Masakatsu, U. Chem. Pharm. Bull. 1997, 45, 719-721.), such as CuI and a base, such as potassium carbonate to provide VII. Halogenation of VII with an appropriate reagent, such as an N-halosuccinimide, would generate intermediate VIII (X═Cl, Br or I, depending on choice of reagent). Subsequent treatment of VIII with a base, such as sodium methoxide, (Wolfe, J. et al. J. Am. Chem. Soc. 1980, 102, 3646-3647) affords the cyclized intermediate IX. Finally, sequential treatment of IX with an equimolar amount of a base, such as lithium diisopropylamide, followed by an equimolar amount of an appropriate R₃ alkylating agent, such as R₃—Br, then a second equimolar amount of a base, such as lithium diisopropylamide, followed by an equimolar amount of an appropriate R₄ alkylating agent, such as R₄—Br, results in the formation of desired compound of formula I, or an intermediate which could be further transformed by methods known in the art to the desired compound of formula I

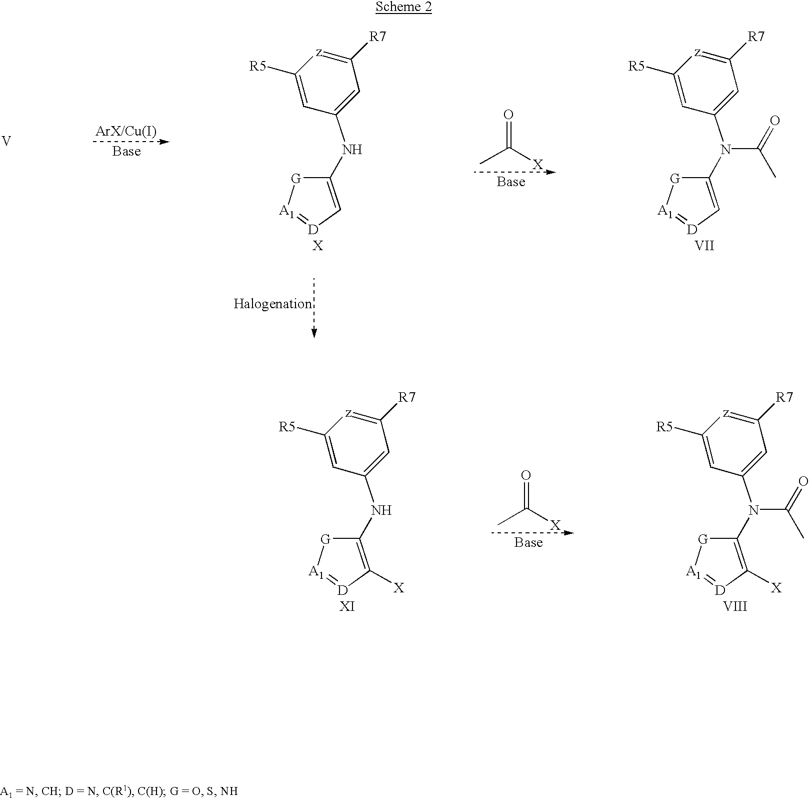

An alternative synthesis of intermediates VII and VIII is described in Scheme 2. Treatment of amine V with an appropriate aryl halide, in the presence of a copper reagent, such as CuI and a base, such as potassium carbonate provides intermediate X. Subsequently, acetylation of X with an appropriate reagent, such as acetyl chloride in the presence of a base, such as diisopropylethylamine, affords VII. On the other hand, halogenation of X with an appropriate reagent, such as N-bromosuccinimide would generate intermediate XI (X=halogen), which upon acetylation with an appropriate reagent, such as acetyl chloride in the presence of a base, such as diisopropylethylamine affords intermediate VIII. Further synthetic elaboration of intermediates VII or VIII, as described in Scheme 1, would lead to the formation of the desired compound of formula I, or an intermediate which could be further transformed by methods known in the art to the desired compound of formula I.

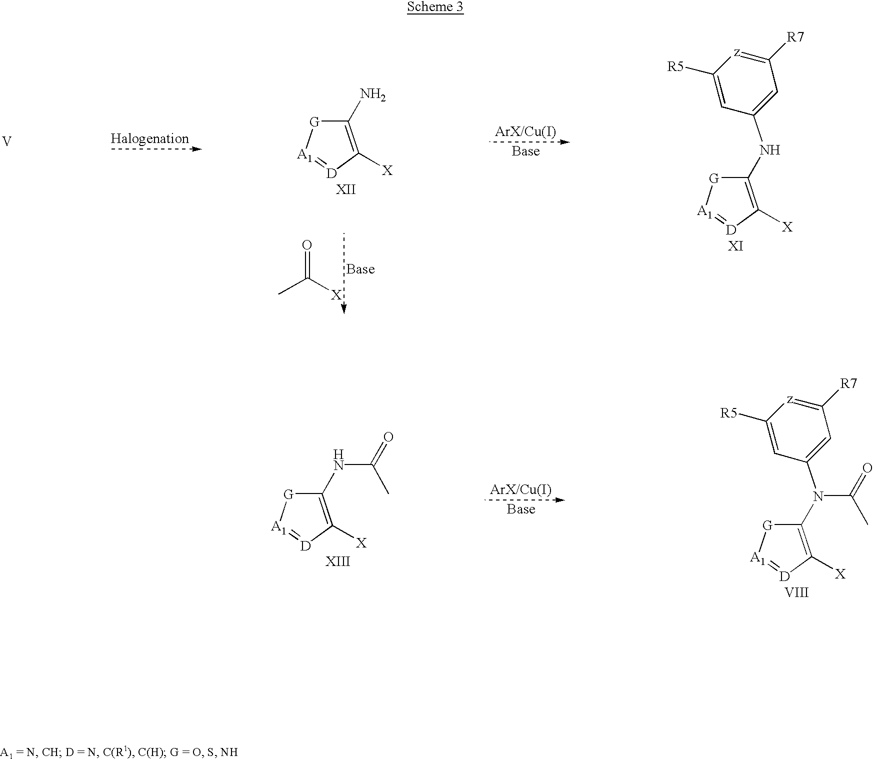

Another method for preparing intermediates XI and VIII is described in Scheme 3. Halogenation of V with an appropriate reagent, such as N-bromosuccinimide would generate intermediate XII (X=halogen), which upon treatment with an appropriate aryl halide, preferably an aryl iodide, in the presence of a copper reagent, such as CuI and a base, such as potassium carbonate provides intermediate XI. Alternatively, acetylation of XII with an appropriate reagent, such as acetyl chloride in the presence of a base, such as diisopropylethylamine affords intermediate XIII. Treatment with an appropriate aryl halide, preferably an aryl iodide, in the presence of a copper reagent, such as CuI and a base, such as potassium carbonate provides intermediate VIII. Further synthetic elaboration of intermediates XI, as described in Scheme 2, and VIII, as described in Scheme 1, would lead to the formation of the desired compound of formula I, or an intermediate which could be further transformed by methods known in the art to the desired compound of formula I.

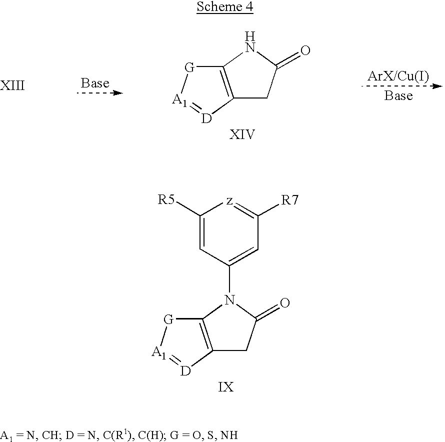

An alternative synthesis of intermediate IX from precursor XIII is delineated in Scheme 4. Treatment of XIII with an appropriate base, such as sodium methoxide would produce cyclized intermediate XIV which upon exposure to an appropriate aryl halide in the presence of a copper reagent, such as CuI and a base, such as potassium carbonate provides intermediate IX. Further synthetic elaboration of IX, as described in Scheme 1, would lead to the formation of the desired compound of formula I, or an intermediate which could be further transformed by methods known in the art to the desired compound of formula I.

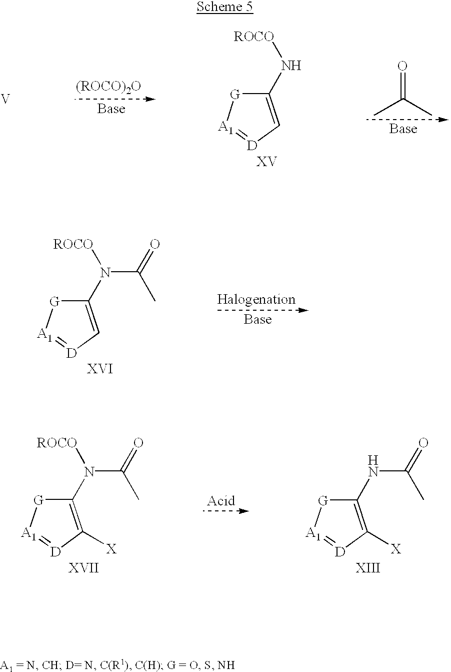

An alternative synthesis of intermediate XIII, using an ortho-metalation directing group, is delineated in Scheme 5. Treatment of amine V with an appropriate acylating agent, such as (t-BOC)₂O in the presence of a base, such as diisopropylethylamine, affords intermediate XV which upon acetylation with an appropriate reagent, such as acetyl chloride in the presence of a base, such as diisopropylethylamine would generate intermediate XVI. Halogenation of XVI with an appropriate reagent, such as N-bromosuccinimide, in the presence of a base, such as n-butyllithium (Konoike, T.; Kanda, Y.; Araki, Y. Tetrahedron Lett. 1996, 37, 3339-3342) at a suitable temperature, such as −78° C. to 0° C., preferably −78° C. would generate intermediate XVII. Finally, treatment of XVII with an acid, such as trifluoroacetic acid, yields the intermediate XIII. Further synthetic elaboration of intermediate XIII, as described in Scheme 3, would lead to the formation of the desired compound of formula I, or an intermediate which could be further transformed by methods known in the art to the desired compound of formula I.

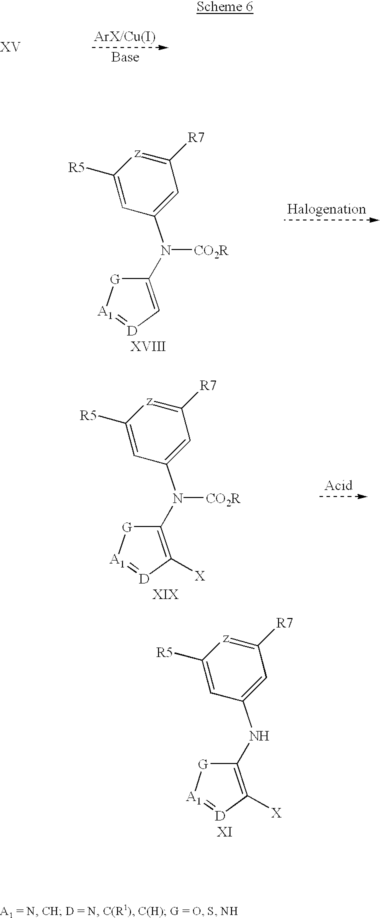

Scheme 6 describes an alternative synthesis of intermediate XI. Treatment of intermediate XV with an appropriate aryl halide, in the presence of a copper reagent, such as CuI and a base, such as potassium carbonate would provide intermediate XVIII. Subsequently, halogenation of XVIII with an appropriate reagent, such as N-bromosuccinimide, in the presence of a base, such as tert-butyllithium would generate intermediate XIX (X=halogen), which upon treatment with an acid, such as trifluoroacetic acid, would yield the intermediate XI. Further synthetic elaboration of intermediate XI, as described in Scheme 2, would lead to the formation of the desired compound of formula I, or an intermediate which could be further transformed by methods known in the art to the desired compound of formula I.

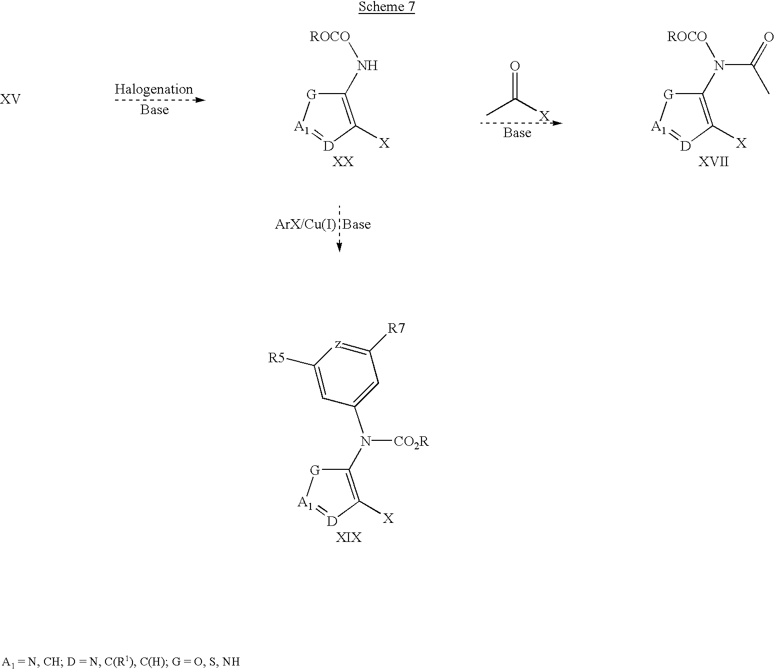

An alternative synthesis of intermediates XVII and XIX is described in Scheme 7. Halogenation of XV with an appropriate reagent, such as N-bromosuccinimide, in the presence of a base, such as tert-butyllithium would generate intermediate XX. Subsequently, acetylation with an appropriate reagent, such as acetyl chloride in the presence of a base, such as diisopropylethylamine would generate intermediate XVII. On the other hand, treatment of XX with an appropriate aryl halide, preferably an aryl iodide, in the presence of a copper reagent, such as CuI and a base, such as potassium carbonate would provide intermediate XIX. Further synthetic elaboration of intermediates XVII, as described in Scheme 5, and XIX, as described in Scheme 6, leads to formation of the desired compound of formula I, or an intermediate which could be further transformed by methods known in the art to the desired compound of formula I.

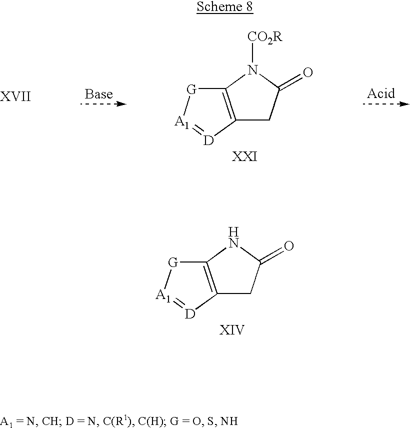

An alternative preparation of intermediate XIV is described in Scheme 8. Treatment of XVII in the presence of a base, such as sodium methoxide affords the cyclized intermediate XXI, which upon exposure to an acid, such as trifluoroacetic acid provides the intermediate XIV. Further synthetic elaboration of intermediate XIV, as described in Scheme 4, leads to the formation of the desired compound of formula I, or an intermediate which could be further transformed by methods known in the art to the desired compound of formula I.

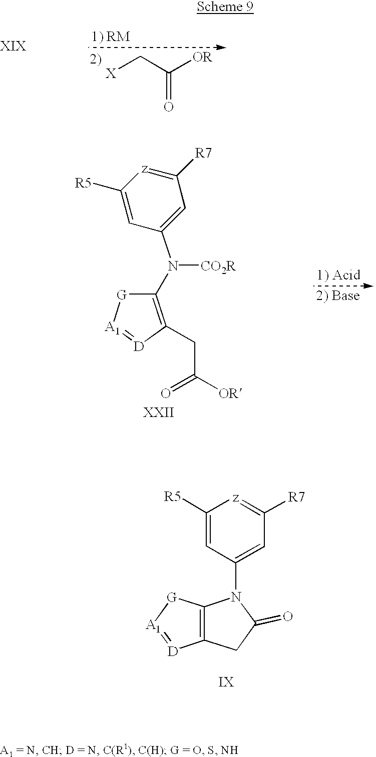

An alternative synthesis of IX is described in Scheme 9. Treatment of intermediate XIX under metal-halogen exchange conditions, using an appropriate metal reagent RM, wherein M can be Li or Mg, such as cyclopentylmagnesium bromide followed by alkylation of the resultant aryl metal species with an alkyl haloacetate, preferably methyl bromoacetate, to provide intermediate XXII. Finally, treatment of XXII with an acid, such as trifluoroacetic acid followed by exposure of the mixture to a base, such as sodium methoxide would result in the formation of cyclized intermediate IX. Further synthetic elaboration of intermediate IX, as described in Scheme 1, would yield the desired compound of formula I, or an intermediate which could be further transformed by methods known in the art to the desired compound of formula I.

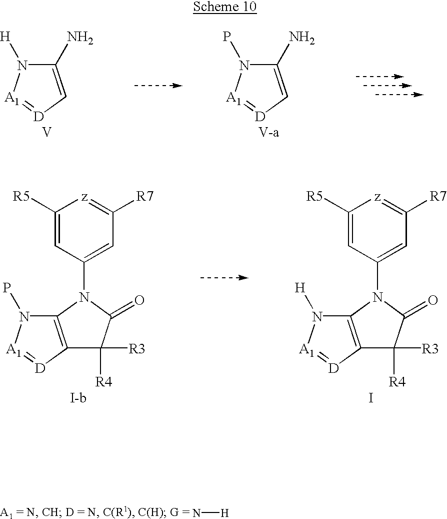

One skilled in the art will recognize the need to use, on occasion, protecting groups on the starting amino heterocycles (Scheme 10). Such a protection scheme would prevent undesired reaction at certain sites, for example, when in V G=NH, a protecting group such as a benzyloxycarbonyl, can be employed to temporarily protect the nitrogen functionality to produce protected amine V-a (P=protecting group). Upon completion of the synthetic transformations leading to the protected form (I-b; G=N—H) of the desired compound of formula I, simple removal of the protecting group using appropriate conditions, for example for a benzyloxycarbonyl, palladium on charcoal under an atmosphere of hydrogen, would yield the desired compound of formula I, or an intermediate which could be further transformed by methods known in the art to the desired compound of formula I. Such protections and deprotections can easily be carried out by one skilled in the art and are well known in the literature.

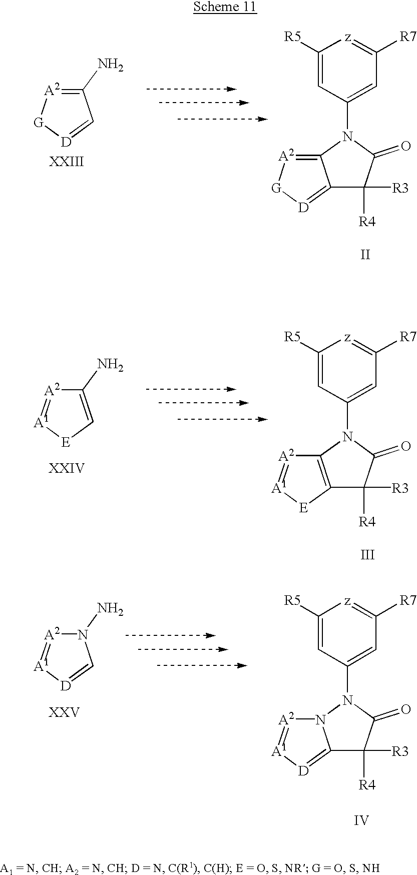

One skilled in the art will recognize that compounds of general formula II, III and IV can also be prepared from the appropriate amino heterocycles, XXIII, XXIV and XXV respectively, using methods analogous to those described above for synthesizing compounds of general formula I.

Analogs of compounds of formulas I, II, III and IV, wherein the carbonyl is replaced by a thiocarbonyl, exemplified by but not limited to analogs of formula I shown here, can be obtained via treatment of I with an appropriate thionating reagent, such as P₄S₁₀, in a high boiling solvent, such as tetralin.

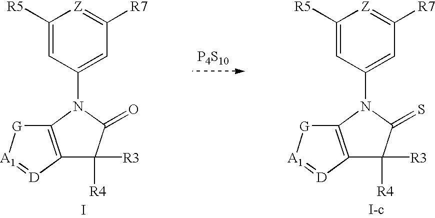

Analogs of compounds of formulas I, II and IV, wherein D is a carbon substituted with various groups, for example, but not limited to, halogen, CN, CHO, an alkyl group, an alkyl or aryl sulfide, sulfoxide or sulfone may be prepared as described below. As exemplified in Scheme 12 for compounds of formula I, halogenation with an N-halosuccinimide, such as N-bromosuccinimide, would result in the formation of intermediate XXVI. Subsequent metal-halogen exchange of the halide with an organometallic reagent RM, such as cyclopentylmagnesium bromide, affords intermediate XXVII (wherein M can be Li or Mg). Treatment of the latter with an electrophilic reagent E+ capable of transferring a functional group, provides the analog XXVIII wherein E can be, for example, but not restricted to, R¹, OR¹, CN, COR¹, S(O)R¹, SO₂R¹.

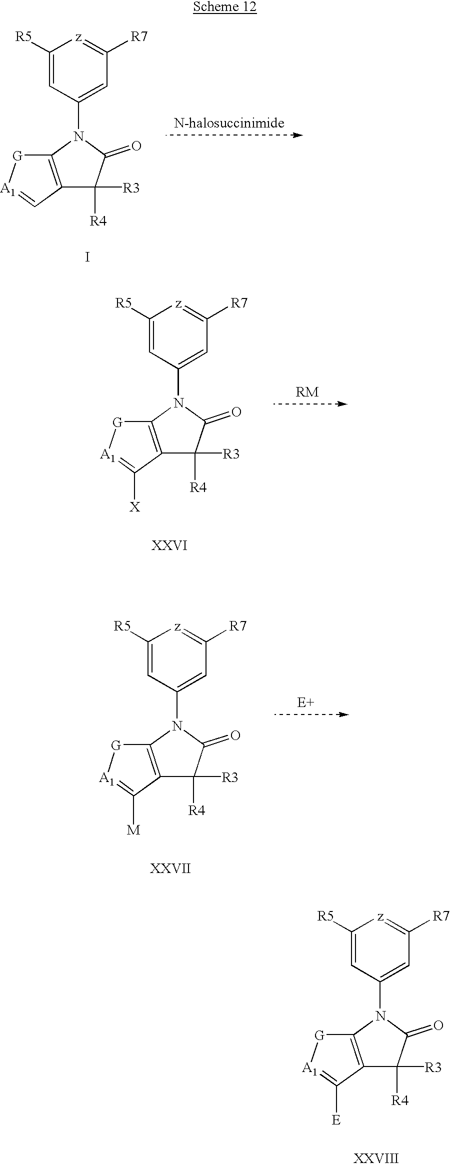

Analogs of III wherein E is a nitrogen substituted with various groups, for example, but not limited to, CHO, an alkyl group, an alkyl or aryl sulfoxide or sulfone, may be prepared as described below and outlined in Scheme 13. Sequential treatment of III with a base, such as lithium diisopropylamide, and an electrophilic reagent E+ capable of transferring a functional group, provides XXIX wherein E can be, for example, but not restricted to, R¹, COR¹, S(O)R¹, SO₂R¹.

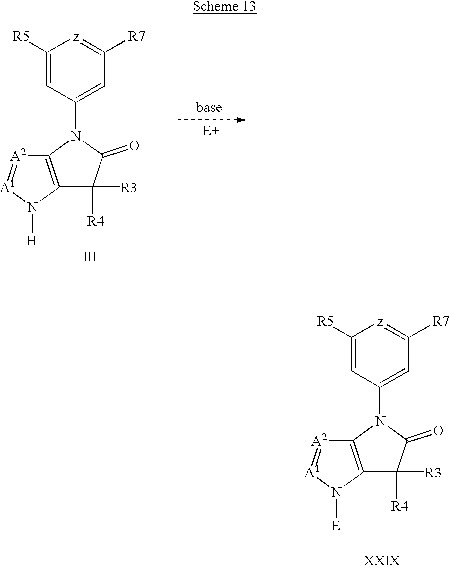

The compounds of general formula I are listed in Table 1 along with the corresponding, starting amino heterocycle. Starting amino heterocycles may be prepared by methods known to those skilled in the art. An exemplary reference is provided for each amino heterocycle.

TABLE 1

| | | |
|---|---|---|
| 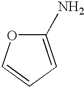 | → | 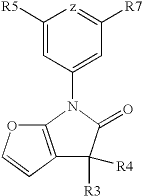 |
| Arch. Pharm. (Weinheim Ger.), 1975, | | |
| 308, 713. | | |
| 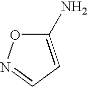 | → | 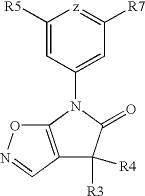 |
| Chem. Pharma, Bull. 1966, 14, 1277. | | |
| 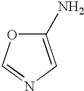 | → | 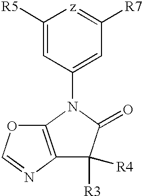 |
| Univ. California, Santa Barbara, CA, | | |
| USA. Avail. Univ. Microfilms Int., | | |
| Order No. DA8428613. (1984) | | |
| 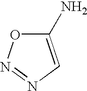 | → | 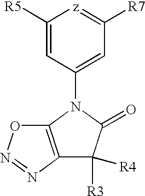 |
| Khim. Geterotsikl. Soedin., USSR, 1980, | | |
| 9, 1244. | | |
| 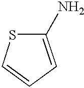 | → | 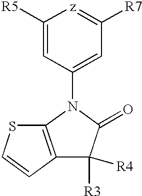 |
| Synthesis, 1977, 255; | | |
| J. Heterocycl. Chem. 1978, 15, 81. | | |
| 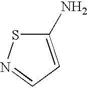 | → | 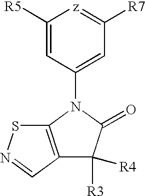 |
| Ger. Offen. Germany, 1998, 8. | | |
| 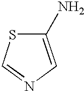 | → | 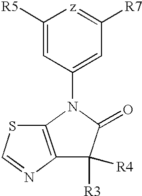 |
| Can. J. Chem., 1986, 64, 1102. | | |
| 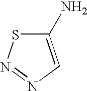 | → | 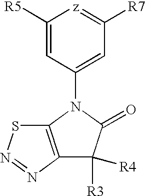 |
| Heterocycles, 1997, 44, 197. | | |
| 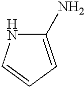 | → | 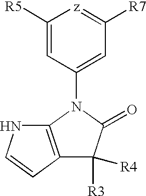 |
| Tetrahedron Lett., 1995, 36, 9261. | | |
| 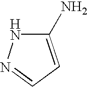 | → | 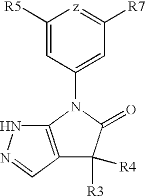 |
| J. Chem. Soc. Perkin Trans. 1, 1990, 809. | | |
| 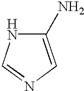 | → | 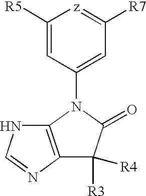 |
| J. Chem. Soc. Perkin Trans. 1, 1980, | | |
| 2316. | | |
| 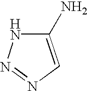 | → | 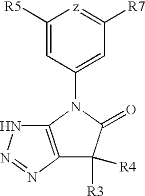 |
| J. Chem. Soc. C., 1971, 1501, 1504, 1507; | | |
| Tetrahedron Lett., 1973, 1137. | | |

The compounds of general formula II are listed in Table 2. Starting amino heterocycles may be prepared by methods known to those skilled in the art. An exemplary reference is provided for each amino heterocycle.

TABLE 2

| | | |
|---|---|---|
| 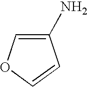 | → | 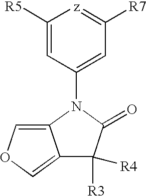 |
| Bull. Soc. Chim. Fr. 1994, 131, 429. | | |
| 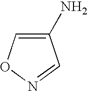 | → | 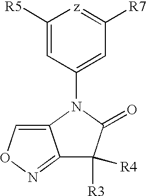 |
| J. Org. Chem., 1987, 52, 2714. | | |
| 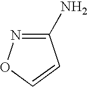 | 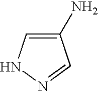 | 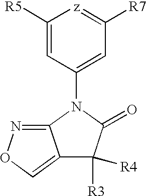 |
| Chem. Phar. Bull., 1966, 14, 1277. | | |
| 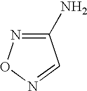 | → | 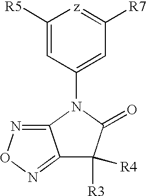 |
| J. Gen. Chem. USSR (Engl. Transl.), | | |
| 1992, 62, 2236. | | |
| 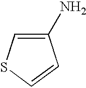 | → | 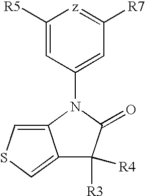 |
| J. Heterocycl. Chem., 1984, 21, 393. | | |
| 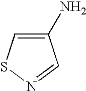 | → | 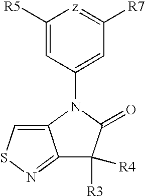 |
| J. Chem. Soc., 1959, 3061. | | |
| 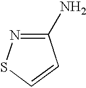 | → | 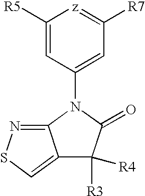 |
| J. Chem. Soc., 1965, 7277. | | |
| 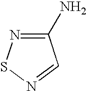 | → | 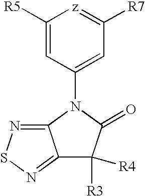 |
| J. Org. Chem., 1967, 32, 2823. | | |
| 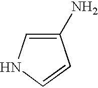 | → | 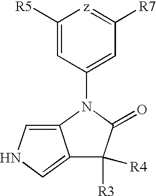 |
| Farmaco, Ed. Sci. 1984, 39, 538. | | |
| 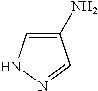 | → | 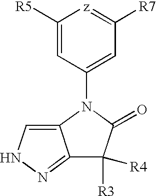 |
| Pharmazie, 1999, 54, 705. | | |
| 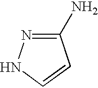 | → | 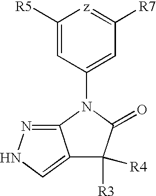 |
| Commercially available. | | |
| 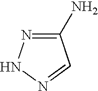 | → | 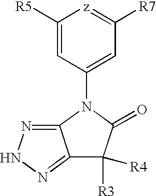 |
| Adv. Heterocycl. Chem., 1986, 40, 129. | | |

The compounds of general formula III are listed in Table 3. Starting amino heterocycles may be prepared by methods known to those skilled in the art. An exemplary reference is provided for each amino heterocycle.

TABLE 3

| | | |
|---|---|---|
| 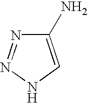 | → | 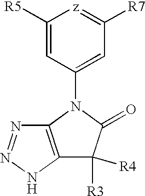 |
| J. Am. Chem. Soc 1956, 78, 5832. | | |
| 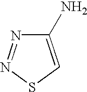 | → | 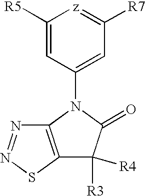 |
| J. Chem. Soc. 1965, 5166. | | |
| 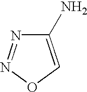 | → | 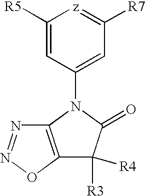 |
| Khim. Geterotsikl. Soedin. 1987, 2, 175. | | |
| 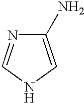 | → | 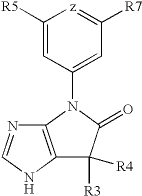 |
| J. Chem. Soc. Perkin1 1992, 2779. | | |
| 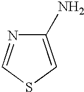 | → | 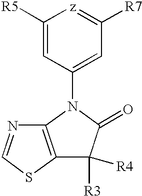 |
| Commercially Available | | |
| 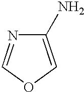 | → | 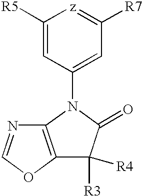 |
| J. Chem. Soc. 1965, 5166. | | |
| 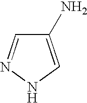 | → | 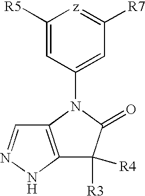 |
| J. Chem. Soc. 1925, 2939. | | |
| 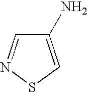 | → | 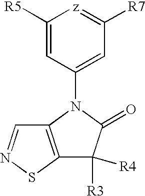 |
| J. Chem. Soc. 1959, 3061. | | |
| 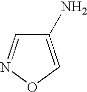 | → | 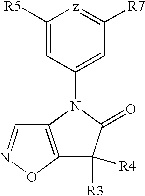 |
| J. Org. Chem. 1987, 52, 2714. | | |
| 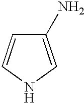 | → | 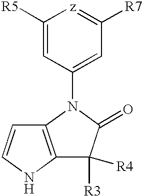 |
| Farmaco, Ed. Sci. 1984, 39, 538. | | |
| 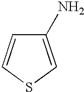 | → | 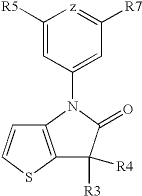 |
| Eur. J. Med. Chem. Chim. Ther. 1991, 26, | | |
| 3. | | |
| 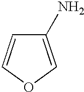 | → | 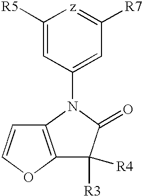 |
| J. Am. Chem. Soc., 1984, 106, 5753. | | |

The compounds of general formula IV are listed in Table 4. Starting amino heterocycles may be prepared by methods known to those skilled in the art. An exemplary reference is provided for each amino heterocycle.

TABLE 4

| | | |
|---|---|---|
| 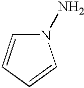 | → | 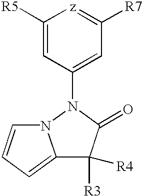 |
| Commercially available | | |
| 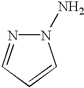 | → | 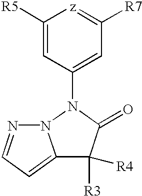 |
| Tetrahedron Lett. 1985, 44, 5485. | | |
| 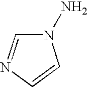 | → | 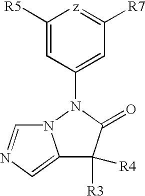 |
| Synthesis, GE, 1982, 7, 592. | | |
| 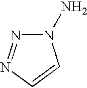 | → | 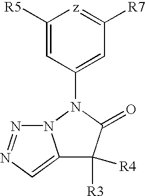 |
| Heterocycles, 1998, 48, 695. | | |
| 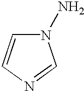 | → | 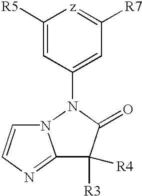 |
| Synthesis, GE, 1982, 7, 592. | | |
| 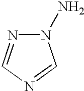 | → | 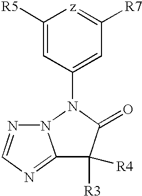 |
| Synthesis, 1989, 4, 269. | | |
| 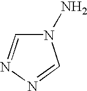 | → | 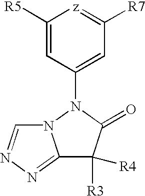 |
| Commercially available | | |
| 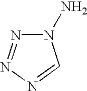 | → | 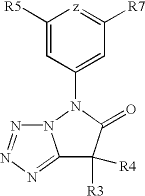 |
| Chem. Heterocycl. Compd. 1983, 19, 681. | | |

Resolution of Enantiomers

There are several ways to resolve the compounds of the invention into their enantiomerically pure forms. One such method is chiral HPLC. An exemplary column packing is Chiracel-OD (Diacel Chemistry Industries). An exemplary solvent system is 9:1 hexanes: iso-propyl alcohol.

Description of Biological Properties

The biological properties of representative compounds of the formula I, II, III or IV may be investigated by way of the experimental protocol described below. Preferred compounds will have K_{d} values<10 μM.

Assay to Determine Inhibition of LFA-1 Binding to ICAM-1

Purpose of Assay:

This assay protocol is designed to study the direct antagonism, by a test compound, of the interaction of the CAM, ICAM-1 with the Leukointegrin CD 18/CD11a (LFA-1).

Description of Assay Protocol:

LFA-1 is immunopurified using the TS2/4 antibody from a 20 g pellet of human JY or SKW3 cells, utilizing a protocol previously described (Dustin, M. J.; et al., J. Immunol. 1992, 148, 2654-2660). The LFA-1 is purified from SKW3 lysates by immunoaffinity chromatography on TS2/4 LFA-1 mAb Sepharose and eluted at pH 11.5 in the presence of 2 mM MgCl₂ and 1% octylglucoside. After collection and neutralization of fractions from the TS2/4 column, samples are pooled and precleared with Protein G agarose.

A soluble form of ICAM-1 is constructed, expressed, purified and characterized as previously described (Marlin, S.; et al., Nature, 1990, 344, 70-72 and see Arruda, A.; et al., Antimicrob. Agents Chemother. 1992, 36, 1186-1192). Briefly, isoleucine 454 which is located at the putative boundary between domain 5 of the ectodomain and the transmembrane domain, is changed to a stop codon using standard oligonucleotide-directed mutagenesis. This construction yields a molecule identical with the first 453 amino acids of membrane bound ICAM-1. An expression vector is created with a hamster dihydrofolate reductase gene, a neomycin-resistance marker, and the coding region of the sICAM-1 construct described above, along with the promoter, splice signals, and polyadenylation signal of the SV40 early region. The recombinant plasmid is transfected into CHO DUX cells using standard calcium phosphate methods. Cells are passaged in selective media (G418) and colonies secreting sICAM-1 are amplified using methotrexate. sICAM-1 is purified from serum-free media using traditional non-affinity chromatographic techniques, including ion exchange and size exclusion chromatography.

LFA-1 binding to ICAM-1 is monitored by first incubating sICAM-1 at 40 μg/mL in Dulbecco's phosphate buffered saline with calcium and magnesium, additional 2 mM MgCl₂ and 0.1 mM PMSF (Diluting Buffer) in a 96-well plate for 30 min at room temperature. Plates are then blocked by the addition of 2% (w/v) bovine serum albumin in Diluting Buffer for 37° C. for 1 h. Blocking solution is removed from wells, and test compounds are diluted and then added followed by the addition of approximately 25 ng of immunoaffinity purified LFA-1. The LFA-1 is incubated in the presence of test compound and ICAM-1 at 37° C. for 1 h. Wells are washed 3 times with Diluting Buffer. The bound LFA-1 is detected by the addition of a polyclonal antibody directed against a peptide corresponding to the CD18 cytoplasmic tail in a 1:100 dilution with Diluting Buffer and 1% BSA and allowed to incubate for 45 min at 37° C. Wells are washed 3 times with Diluting Buffer and the bound polyclonal antibody is detected by the addition of a 1:4000 dilution of horse radish peroxidase conjugated to goat immunoglobulin directed against rabbit immunoglobulin. This reagent is allowed to incubate for 20 min at 37° C., wells are washed as above and the substrate for the horse radish peroxidase is added to each well to develop a quantitative colorimetric signal proportional to the amount of LFA-1 bound to sICAM-1. Soluble ICAM-1 (60 μg/mL) is used as a positive control for inhibition of the LFA-1/ICAM-1 interaction. The lack of the addition of LFA-1 to the binding assay is used as a background control for all samples. A dose-response curve is obtained for all test compounds.

Description of Therapeutic Use

The novel small molecules of formula I, II, III or IV provided by the invention inhibit the ICAM-1/LFA-1 dependent homotypic aggregation of human lymphocytes and human lymphocyte adherence to ICAM-1. These compounds have therapeutic utility in the modulation of immune cell activation/proliferation, e.g., as competitive inhibitors of intercellular ligand/receptor binding reactions involving CAMs and Leukointegrins. To be more specific, the compounds of the invention may be used to treat certain inflammatory conditions, including conditions resulting from a response of the non-specific immune system in a mammal (e.g., adult respiratory distress syndrome, shock, oxygen toxicity, multiple organ injury syndrome secondary to septicemia, multiple organ injury syndrome secondary to trauma, reperfusion injury of tissue due to cardiopulmonary bypass, myocardial infarction or use with thrombolysis agents, acute glomerulonephritis, vasculitis, reactive arthritis, dermatosis with acute inflammatory components, stroke, thermal injury, hemodialysis, leukapheresis, ulcerative colitis, necrotizing enterocolitis and granulocyte transfusion associated syndrome) and conditions resulting from a response of the specific immune system in a mammal (e.g., psoriasis, organ/tissue transplant rejection, graft vs. host reactions and autoimmune diseases including Raynaud's syndrome, autoimmune thyroiditis, dermatitis, multiple sclerosis, rheumatoid arthritis, insulin-dependent diabetes mellitus, uveitis, inflammatory bowel disease including Crohn's disease and ulcerative colitis, and systemic lupus erythematosus). The compounds of the invention may also be used in treating asthma or as an adjunct to minimize toxicity with cytokine therapy in the treatment of cancers. In general these compounds may be employed in the treatment of those diseases currently treatable through steroid therapy.

Thus, another aspect of the invention is the provision of a method for the treatment or prophylaxis of the above-described conditions through the adminstration of therapeutic or prophylactic amounts of one or more compounds of the formula I.

In accordance with the method provided by the invention, the novel compounds of formula I, II, III or IV may be administered for either a prophylactic or therapeutic purpose either alone or with other immunosuppressive or antiinflammatory agents. When provided prophylactically, the immunosuppressive compound(s) are provided in advance of any inflammatory response or symptom (for example, prior to, at, or shortly after the time of an organ or tissue transplant but in advance of any symptoms of organ rejection). The prophylactic administration of a compound of the formula I, II, III or IV serves to prevent or attenuate any subsequent inflammatory response (such as, for example, rejection of a transplanted organ or tissue, etc.). The therapeutic administration of a compound of the formula I, II, III or IV serves to attenuate any actual inflammation (such as, for example, the rejection of a transplanted organ or tissue). Thus, in accordance with the invention, a compound of the formula I, II, III or IV can be administered either prior to the onset of inflammation (so as to suppress an anticipated inflammation) or after the initiation of inflammation.

The novel compounds of the formula I, II, III or IV may, in accordance with the invention, be administered in single or divided doses by the oral, parenteral or topical routes. A suitable oral dosage for a compound of formula I, II, III or IV would be in the range of about 0.1 mg to 10 g per day. In parenteral formulations, a suitable dosage unit may contain from 0.1 to 250 mg of said compounds, whereas for topical administration, formulations containing 0.01 to 1% active ingredient are preferred. It should be understood, however, that the dosage administration from patient to patient will vary and the dosage for any particular patient will depend upon the clinician's judgement, who will use as criteria for fixing a proper dosage the size and condition of the patient as well as the patient's response to the drug.

When the compounds of the present invention are to be administered by the oral route, they may be administered as medicaments in the form of pharmaceutical preparations which contain them in association with a compatible pharmaceutical carrier material. Such carrier material can be an inert organic or inorganic carrier material suitable for oral administration. Examples of such carrier materials are water, gelatin, talc, starch, magnesium stearate, gum arabic, vegetable oils, polyalkylene-glycols, petroleum jelly and the like.

The pharmaceutical preparations can be prepared in a conventional manner and finished dosage forms can be solid dosage forms, for example, tablets, dragees, capsules, and the like, or liquid dosage forms, for example solutions, suspensions, emulsions and the like. The pharmaceutical preparations may be subjected to conventional pharmaceutical operations such as sterilization. Further, the pharmaceutical preparations may contain conventional adjuvants such as preservatives, stabilizers, emulsifiers, flavor-improvers, wetting agents, buffers, salts for varying the osmotic pressure and the like. Solid carrier material which can be used include, for example, starch, lactose, mannitol, methyl cellulose, microcrystalline cellulose, talc, silica, dibasic calcium phosphate, and high molecular weight polymers (such as polyethylene glycol).

For parenteral use, a compound of formula I, II, III or IV can be administered in an aqueous or non-aqueous solution, suspension or emulsion in a pharmaceutically acceptable oil or a mixture of liquids, which may contain bacteriostatic agents, antioxidants, preservatives, buffers or other solutes to render the solution isotonic with the blood, thickening agents, suspending agents or other pharmaceutically acceptable additives. Additives of this type include, for example, tartrate, citrate and acetate buffers, ethanol, propylene glycol, polyethylene glycol, complex formers (such as EDTA), antioxidants (such as sodium bisulfite, sodium metabisulfite, and ascorbic acid), high molecular weight polymers (such as liquid polyethylene oxides) for viscosity regulation and polyethylene derivatives of sorbitol anhydrides. Preservatives may also be added if necessary, such as benzoic acid, methyl or propyl paraben, benzalkonium chloride and other quaternary ammonium compounds.

The compounds of this invention may also be administered as solutions for nasal application and may contain in addition to the compounds of this invention suitable buffers, tonicity adjusters, microbial preservatives, antioxidants and viscosity-increasing agents in an aqueous vehicle. Examples of agents used to increase viscosity are polyvinyl alcohol, cellulose derivatives, polyvinylpyrrolidone, polysorbates or glycerin. Microbial preservatives added may include benzalkonium chloride, thimerosal, chloro-butanol or phenylethyl alcohol.

Additionally, the compounds provided by the invention can be administered topically or by suppository.

Formulations

Compounds of the formula I, II, III or IV can be formulated for therapeutic administration in a number of ways. Descriptions of several exemplary formulations are given below.

### EXAMPLE A

Capsules or Tablets

| Example A-1 | | Example A-2 | |
|---|---|---|---|
| Ingredients | Quantity | Ingredients | Quantity |
| Compound of formula | 250 mg | Compound of formula I, | 50 mg |
| I, II, III or IV | | II, III or IV | |
| Starch | 160 mg | Dicalcium Phosphate | 160 mg |
| Microcrys. Cellulose | 90 mg | Microcrys. Cellulose | 90 mg |
| Sodium Starch | 10 mg | Stearic acid | 5 mg |
| Glycolate | | | |
| Magnesium Stearate | 2 mg | Sodium Starch Glycolate | 10 mg |
| Fumed colloidal silica | 1 mg | Fumed colloidal silica | 1 mg |

The compound of formula I, II, III or IV is blended into a powder mixture with the premixed excipient materials as identified above with the exception of the lubricant. The lubricant is then blended in and the resulting blend compressed into tablets or filled into hard gelatin capsules.

### EXAMPLE B

Parenteral Solutions

| Ingredients | Quantity |
|---|---|
| Compound of formula I, II, III | 500 mg |
| or IV | |
| PEG 400 | 40% by volume |
| Ethyl Alcohol | 5% by volume |
| Saline | 55% by volume |

The excipient materials are mixed and then added to one of the compounds of formula I, II, III or IV in such volume as is necessary for dissolution. Mixing is continued until the solution is clear. The solution then filtered into the appropriate vials or ampoules and sterilized by autoclaving.

### EXAMPLE C

Suspension

| Ingredients | Quantity |
|---|---|
| Compound of formula I, II, III | 100 mg |
| or IV | |
| Citric acid | 1.92 g |
| Benzalkonium chloride | 0.025% by weight |
| EDTA | 0.1% by weight |
| Polyvinylalcohol | 10% by weight |
| Water | q.s. to 100 mL |

The excipient materials are mixed with the water and thereafter one of the compounds of formula I, II, III or IV is added and mixing is continued until the suspension is homogeneous. The suspension is then transferred into the appropriate vials or ampoules.

### EXAMPLE D

Topical Formulation

| Ingredients | Quantity |
|---|---|
| Compound of formula I, II, III | 5% by weight |
| or IV | |
| Tefose 63 | 13% by weight |
| Labrafil M 1944 CS | 3% by weight |
| Paraffin Oil | 8% by weight |
| Methylparaben (MP) | 0.15% by weight |
| Propylparaben (PP) | 0.05% by weight |
| Deionized water | q.s. to 100 |

The proper amounts of Tefose 63, Labrafil M 1944 CS, Paraffin oil and water are mixed and heated at 75° C. until all components have melted. The mixture is then cooled to 50° C. with continuous stirring. Methylparaben and propylparaben are added with mixing and the mixture is cooled to ambient temperature. The compound of formula I, II, III or IV is added to the mixture and blended well.

What is claimed is:

1. A compound of the formula I, II, III or IV 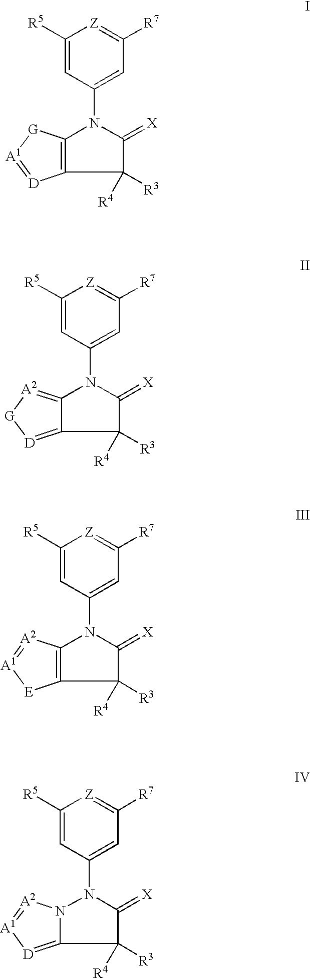
wherein:
A¹ is ═N—, ═C(H)—, or ═C(R′)— wherein R′ is halogen, —CN, —Oalkyl, —CO₂alkyl or —SO₂alkyl, wherein the foregoing alkyl moieties are of 1 to 3 carbon atoms;
A² is ═N— or ═C(H)—;
D is ═N—, ═C(R¹)—, ═C(H)—, ═C(SO₂R¹)—, ═C(S(O)R¹)—, ═C(C(O)R¹)—, ═C(C(O)H)—, ═C(SR^{1a})—, ═C(OR^{1a})— or ═C(NHR^{1a})—,
wherein R¹ is selected from the class consisting of:
(A) —R¹⁰⁰, which is:
branched or unbranched alkyl of 1 to 6 carbon atoms, alkenyl of 2 to 6 carbon atoms or cycloalkyl or cycloalkenyl of 3 to 6 carbon atoms, in which alkyl, alkenyl, cycloalkyl or cycloalkenyl group one or more hydrogen atoms are optionally and independently replaced with:
(i) halogen,
(ii) oxo,
(iii) aryl or heteroaryl which is selected from the class consisting of phenyl, naphthyl, indolyl, thiophenyl, pyridyl, pyrimidinyl, furyl, pyrrolyl, oxazolyl, thiazolyl, pyrazolyl, isoxazolyl, imidazolyl, isothiazolyl, oxadiazolyl, triazolyl, thiadiazolyl, pyridazinyl, pyrazinyl, triazinyl, indolyzinyl, isoindolyl, benzo[b]furanyl, benzo[b]thiophenyl, indazolyl, benzthiazolyl, benzimidazolyl, quinolinyl, isoquinolinyl, purinyl, quinolizinyl, cinnolinyl, pthalaninyl, quinoxalinyl, napthyridinyl, pteridinyl and quinazolinyl, wherein one or more hydrogen atoms of said aryl or heteroaryl group are optionally and independently replaced with:
(a) alkyl of 1 to 3 carbon atoms,
(b) —COOH,
(c) —SO₂OH,
(d) —PO(OH)₂,
(e) a group of the formula —COOR⁸, wherein R⁸ is straight or branched alkyl of 1 to 5 carbon atoms or cycloalkyl of 3 to 5 carbon atoms,
(f) a group of the formula —NR⁹R¹⁰, wherein R⁹ and R¹⁰ are each independently a hydrogen atom, alkyl of 1 to 6 carbon atoms, cycloalkyl of 3 to 6 carbon atoms or acyl of 1 to 7 carbon atoms, or wherein R⁹ and R¹⁰ constitute a saturated hydrocarbon bridge of 3 to 5 carbon atoms which together with the nitrogen atom between them form a heterocyclic ring,
(g) a group of the formula —CONR¹¹R¹², wherein R¹¹ and R¹² are each independently a hydrogen atom, alkyl of 1 to 6 carbon atoms or cycloalkyl of 3 to 6 carbon atoms, or wherein R¹¹ and R¹² constitute a saturated hydrocarbon bridge of 3 to 5 carbon atoms which together with the nitrogen atom between them form a heterocyclic ring, and wherein one carbon atom in said hydrocarbon bridge is optionally replaced by —O—, —S—, S(O)—, SO₂—, —NH—, or —NMe—,
(h) a group of the formula —OR¹³, wherein R¹³ is a hydrogen atom, or an alkyl or acyl group of 1 to 7 carbon atoms,
(i) a group of the formula —SR¹⁴, wherein R¹⁴ is a hydrogen atom, or an alkyl or acyl group of 1 to 7 carbon atoms,
(j) —CN, or
(k) an amidino group of the formula 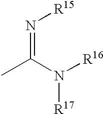
wherein R¹⁵, R¹⁶ and R¹⁷ are each, independently, a hydrogen atom or alkyl of 1 to 3 carbon atoms and wherein two of R¹⁵, R¹⁶ and R¹⁷ may additionally constitute a saturated hydrocarbon bridge of 3 to 5 carbon atoms which together with the nitrogen atom(s) between them form a heterocyclic ring,
(l) halogen,
(m) a group of the formula —NHCONHalkyl, wherein the alkyl moiety contains 1 to 3 carbon atoms,
(n) a group of the formula —NHCOOalkyl, wherein the alkyl moiety contains 1 to 3 carbon atoms,
(iv) a group of the formula —COOR¹⁸, wherein R¹⁸ is straight or branched alkyl of 1 to 7 carbon atoms or cycloalkyl of 3 to 6 carbon atoms,
(v) —CN,
(vi) a group of the formula —CONR¹⁹R²⁰, wherein R¹⁹ and R²⁰ are each, independently, a hydrogen atom, alkyl of 1 to 6 carbon atoms or cycloalkyl of 3 to 6 carbon atoms, or wherein R¹⁹ and R²⁰ constitute a saturated hydrocarbon bridge of 3 to 5 carbon atoms which together with the nitrogen atom between them form a heterocyclic ring, and wherein one carbon atom in said hydrocarbon bridge is optionally replaced by —O—, —S—, S(O)—, SO₂—, —NH—, or —NMe—,
(vii) a group of the formula —OR²¹, wherein R²¹ is a hydrogen atom, or a straight or branched alkyl or acyl group of 1 to 7 carbon atoms, wherein one or more hydrogen atoms of said alkyl or acyl group are optionally replaced with a group independently selected from the class consisting of —OH, —Oalkyl (wherein the alkyl moiety contains 1 to 6 carbon atoms), —NH₂, —NHMe and —NMe₂,
(viii) a group of the formula —SR²², wherein R²² is a hydrogen atom, or an alkyl or acyl group of 1 to 7 carbon atoms, wherein one or more hydrogen atoms of said alkyl or acyl group are optionally replaced with a group independently selected from the class consisting of —OH, —Oalkyl (wherein the alkyl moiety is 1 to 6 carbon atoms), —NH₂, —NHMe and —NMe₂,
(ix) a group of the formula —NR²³R²⁴, wherein R²³ and R²⁴ are each, independently,
(a) a hydrogen atom,
(b) straight or branched alkyl or acyl of 1 to 7 carbon atoms or cycloalkyl of 3 to 7 carbon atoms, wherein said one or more hydrogen atoms of said alkyl or acyl group are optionally replaced with a group independently selected from the class consisting of —OH, —Oalkyl (wherein the alkyl moiety is 1 to 6 carbon atoms), —NH₂, —NHMe and —NMe₂,
(c) a group of the formula —(CH₂)ₘCOOH, wherein m is 0, 1 or 2,
(d) a group of the formula —(CH₂)ₙCOOR²⁵, wherein n is 0, 1 or 2, and wherein R²⁵ is straight or branched alkyl of 1 to 6 carbon atoms, or
(e) a group of the formula —(CH₂)ₙCONHR²⁵, wherein n is 0, 1 or 2, and wherein R²⁵ is straight or branched alkyl of 1 to 6 carbon atoms,
(x) a quaternary group of the formula 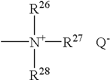
wherein R²⁶, R²⁷ and R²⁸ are each, independently, a branched or unbranched alkyl group of 1 to 7 carbon atoms and Q⁻ is a pharmaceutically acceptable counter ion,
(xi) a saturated, or partially unsaturated heterocyclic group consisting of 3 to 7 ring atoms selected from N, O, C and S, including but not limited to imidazolinyl, imidazolidinyl, pyrrolinyl, pyrrolidinyl, piperidinyl, piperazinyl, morpholinyl, thiomorpholinyl, thiazolidinyl, azepinyl, tetrahydropyranyl, tetrahydrofuranyl, benzodioxolyl, tetrahydrothiophenyl and sulfolanyl, wherein said heterocyclic group is optionally mono- or polysubstituted with oxo, and
(xii) a cycloalkyl group of 3 to 7 carbon atoms,
(B) branched or unbranched carboxylic acid groups of 3 to 6 carbon atoms,
(C) branched or unbranched phosphonic acid groups of 2 to 6 carbon atoms,
(D) branched or unbranched sulfonic acid groups of 2 to 6 carbon atoms,
(E) amidino groups of the formula 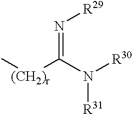
wherein r is 2, 3, 4, 5 or 6, and R²⁹, R³⁰ and R³¹ are each, independently, a hydrogen atom or alkyl of 1 to 3 carbon atoms, and wherein two of R²⁹, R³⁰ and R³¹ may additionally constitute a saturated hydrocarbon bridge of 3 to 5 carbon atoms which together with the nitrogen atom(s) between them form a heterocyclic ring,
(F) guanidino groups of the formula 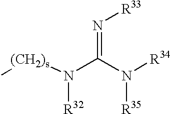
wherein s is 2, 3, 4, 5 or 6, and R³², R³³, R³⁴ and R³⁵ are each, independently, a hydrogen atom or alkyl of 1 to 3 carbon atoms, and wherein two of R³², R³³, R³⁴ and R³⁵ may additionally constitute a saturated hydrocarbon bridge of 3 to 5 carbon atoms which together with the nitrogen atom(s) between them form a heterocyclic ring,
(G) aryl or heteroaryl which is selected from the class consisting of phenyl, naphthyl, indolyl, thiophenyl, pyridyl, pyrimidinyl, furyl, pyrrolyl, oxazolyl, thiazolyl, pyrazolyl, isoxazolyl, imidazolyl, isothiazolyl, oxadiazolyl, triazolyl, thiadiazolyl, pyridazinyl, pyrazinyl, triazinyl, indolyzinyl, isoindolyl, benzo[b]furanyl, benzo[b]thiophenyl, indazolyl, benzthiazolyl, benzimidazolyl, quinolinyl, isoquinolinyl, purinyl, quinolizinyl, cinnolinyl, pthalaninyl, quinoxalinyl, napthyridinyl, pteridinyl and quinazolinyl, wherein one or more hydrogen atoms of said aryl or heteroaryl group are optionally and independently replaced with:
(i) alkyl of 1 to 3 carbon atoms,
(ii) —COOH,
(iii) —SO₂OH,
(iv) —PO(OH)₂,
(v) a group of the formula —COOR³⁶, wherein R³⁶ is straight or branched alkyl of 1 to 5 carbon atoms or cycloalkyl of 3 to 5 carbon atoms,
(vi) a group of the formula —NR³⁷R³⁸, wherein R³⁷ and R³⁸ are each, independently, a hydrogen atom, alkyl of 1 to 6 carbon atoms, cycloalkyl of 3 to 6 carbon atoms or acyl of 1 to 7 carbon atoms, or wherein R³⁷ and R³⁸ constitute a saturated hydrocarbon bridge of 3 to 5 carbon atoms which together with the nitrogen atom between them form a heterocyclic ring,
(vii) a group of the formula —CONR³⁹R⁴⁰, wherein R³⁹ and R⁴⁰ are each, independently, a hydrogen atom, alkyl of 1 to 6 carbon atoms or cycloalkyl of 3 to 6 carbon atoms, or wherein R³⁹ and R⁴⁰ constitute a saturated hydrocarbon bridge of 3 to 5 carbon atoms which together with the nitrogen atom between them form a heterocyclic ring, and wherein one carbon atom in said hydrocarbon bridge is optionally replaced by —O—, —S—, S(O)—, SO₂—, —NH—, or —NMe—,
(viii) a group of the formula —OR⁴¹, wherein R⁴¹ is a hydrogen atom, or an alkyl or acyl group of 1 to 7 carbon atoms,
(ix) a group of the formula —SR⁴², wherein R⁴² is a hydrogen atom, or an alkyl or acyl group of 1 to 7 carbon atoms,
(x) —CN, or
(xi) an amidino group of the formula 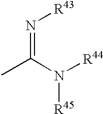
wherein R⁴³, R⁴⁴ and R⁴⁵ are each, independently, a hydrogen atom or alkyl of 1 to 3 carbon atoms, and wherein two of R⁴³, R⁴⁴ and R⁴⁵ may additionally constitute a saturated hydrocarbon bridge of 3 to 5 carbon atoms which together with the nitrogen atom(s) between them form a heterocyclic ring,
(H) groups of the formula —NR⁴⁶R⁴⁷, wherein R⁴⁶ and R⁴⁷ are each independently a hydrogen atom, phenyl which is optionally mono- or polysubstituted with halogen, or R¹⁰⁰, wherein R¹⁰⁰ is as hereinbefore defined,
(I) saturated or unsaturated heterocyclic groups consisting of 3 to 7 ring atoms selected from N, O, C and S, or bicyclic heterocyclic groups consisting of 8 to 11 atoms selected from N, O, C and S, including but not limited to imidazolinyl, imidazolidinyl, pyrrolinyl, pyrrolidinyl, piperidinyl, piperazinyl, morpholinyl, thiomorpholinyl, thiazolidinyl, azepinyl, tetrahydropyranyl, tetrahydrofuranyl, benzodioxolyl, tetrahydrothiophenyl and sulfolanyl, wherein said heterocyclic group is optionally mono- or poly-substituted with moieties selected from the class consisting of:
(i) oxo,
(ii) —OR¹⁰¹, wherein R¹⁰¹ is:
(a) a hydrogen atom,
(b) alkyl of 1 to 7 carbons, wherein any hydrogen atom of said alkyl group is optionally replaced with —OH, —OR¹¹⁰ (wherein R¹¹⁰ is an alkyl moiety of 1 to 6 carbon atoms), —NH₂, —NHMe or —NMe₂,
(c) acyl of 1 to 7 carbons, wherein any hydrogen atom of said acyl group is optionally replaced with —OH, —OR¹¹¹ (wherein R¹¹¹ is an alkyl moiety of 1 to 6 carbon atoms), —NH₂, —NHMe or —NMe₂,
(d) —CONR¹⁰²R¹⁰³, wherein R¹⁰² and R¹⁰³ are each independently a hydrogen atom or alkyl of 1 to 7 atoms, or wherein R¹⁰² and R¹⁰³ constitute a saturated hydrocarbon bridge of 3 to 5 carbon atoms which together with the nitrogen atom between them form a heterocyclic ring, and wherein one carbon atom in said hydrocarbon bridge is optionally replaced by —O—, —S—, S(O)—, SO₂—, —NH—, or —NMe—, or
(e) —COOR¹⁰⁴, wherein R¹⁰⁴ is alkyl of 1 to 7 atoms,
(iii) —CONR¹⁰⁵R¹⁰⁶, wherein R¹⁰⁵ and R¹⁰⁶ are each independently:
(a) a hydrogen atom,
(b) straight or branched alkyl of 1 to 7 atoms or cycloalkyl of 3 to 7 atoms,
(c) benzoyl,
(d) benzyl or
(e) phenyl, wherein said phenyl ring is optionally mono- or polysubstituted with —OR¹¹²,wherein R¹¹² is alkyl of 1 to 6 carbon atoms,
or, wherein R¹⁰⁵ and R¹⁰⁶ constitute a saturated hydrocarbon bridge of 3 to 5 carbon atoms which together with the nitrogen atom between them form a heterocyclic ring, and wherein one carbon atom in said hydrocarbon bridge is optionally replaced by —O—, —S—, S(O)—, SO₂—, —NH—, or —NMe—,
(iv) —COOR¹⁰⁷, wherein R¹⁰⁷ is a hydrogen atom, or straight or branched alkyl of 1 to 7 carbon atoms,
(v) straight or branched alkyl of 1 to 7 carbon atoms, alkenyl or alkynyl of 2 to 7 carbon atoms, or cycloalkyl of 3 to 7 carbons, wherein one or more hydrogen atoms of said alkyl, alkenyl, alkynyl or cycloalkyl group is optionally replaced with a moiety independently selected from the class consisting of:
(a) oxo,
(b) —OH,
(c) —OR¹¹³, wherein R¹¹³ is alkyl of 1 to 6 carbon atoms,
(d) —OCOCH₃,
(e) —NH₂,
(f) —NHMe,
(g) —NMe₂,
(h) —CO₂H, and
(i) —CO₂ R^{1 14} wherein R¹¹⁴ is alkyl of 1 to 3 carbon atoms, or cycloalkyl of 3 to 7 carbons,
(vi) acyl of 1 to 7 carbon atoms, which may be straight, branched or cyclic, and wherein one or more hydrogen atoms of said acyl group is optionally replaced with a moiety independently selected from the class consisting of:
(a) —OH,
(b) —OR¹¹⁵, wherein R¹¹⁵ is alkyl of 1 to 6 carbon atoms,
(c) —NH₂,
(d) —NHMe,
(e) —NMe₂,
(f) —NHCOMe,
(g) oxo,
(h) —CO₂ R¹¹⁶, wherein R¹¹⁶ is alkyl of 1 to 3 carbon atoms,
(i) —CN,
(j) the halogen atoms,
(k) heterocycles selected from the class consisting of imidazolinyl, imidazolidinyl, pyrrolinyl, pyrrolidinyl, piperidinyl, piperazinyl, morpholinyl, thiomorpholinyl, thiazolidinyl, azepinyl, tetrahydropyranyl, tetrahydrofuranyl, benzodioxolyl, tetrahydrothiophenyl and sulfolanyl, and
(l) aryl or heteroaryl selected from the class consisting of phenyl, naphthyl, indolyl, thiophenyl, pyridyl, pyrimidinyl, furyl, pyrrolyl, oxazolyl, thiazolyl, pyrazolyl, isoxazolyl, imidazolyl, isothiazolyl, oxadiazolyl, triazolyl, thiadiazolyl, pyridazinyl, pyrazinyl, triazinyl, indolyzinyl, isoindolyl, benzo[b]furanyl, benzo[b]thiophenyl, indazolyl, benzthiazolyl, benzimidazolyl, quinolinyl, isoquinolinyl, purinyl, quinolizinyl, cinnolinyl, pthalaninyl, quinoxalinyl, napthyridinyl, pteridinyl and quinazolinyl,
(vii) —SO₂R¹⁰⁸, wherein R¹⁰⁸ is:
(a) aryl or heteroaryl which is selected from the group consisting of phenyl, naphthyl, indolyl, thiophenyl, pyridyl, pyrimidinyl, furyl, pyrrolyl, oxazolyl, thiazolyl, pyrazolyl, isoxazolyl, imidazolyl, isothiazolyl, oxadiazolyl, triazolyl, thiadiazolyl, pyridazinyl, pyrazinyl, triazinyl, indolyzinyl, isoindolyl, benzo[b]furanyl, benzo[b]thiophenyl, indazolyl, benzthiazolyl, benzimidazolyl, quinolinyl, isoquinolinyl, purinyl, quinolizinyl, cinnolinyl, pthalaninyl, quinoxalinyl, napthyridinyl, pteridinyl and quinazolinyl, wherein said aryl or heteroaryl moiety is optionally substituted with one or more moieties selected from the class consisting of the halogen atoms, straight or branched alkyl of 1 to 6 carbons, and —OR¹¹⁷ (wherein R¹¹⁷ is hydrogen or alkyl of 1 to 6 carbon atoms),
(b) a heterocyclic group selected from the class consisting of imidazolinyl, imidazolidinyl, pyrrolinyl, pyrrolidinyl, piperidinyl, piperazinyl, morpholinyl, thiomorpholinyl, thiazolidinyl, azepinyl, tetrahydropyranyl, tetrahydrofuranyl, benzodioxolyl, tetrahydrothiophenyl and sulfolanyl, wherein said heterocyclic group is optionally substituted with one or more moieties selected from the class consisting of the halogen atoms, straight or branched alkyl of 1 to 6 carbons, and —OR¹¹⁸ (wherein R¹¹⁸ is hydrogen or alkyl of 1 to 6 carbon atoms), or
(c) straight or branched alkyl of 1 to 7 atoms, wherein said alkyl moiety is optionally substituted with one or more moieties selected from the class consisting of the halogen atoms, straight or branched alkyl of 1 to 6 carbons, and —OR¹¹⁹ (wherein R¹¹⁹ is hydrogen or alkyl of 1 to 6 carbon atoms),
(viii) —COR¹⁰⁹, wherein R¹⁰⁹ is:
(a) aryl or heteroaryl which is selected from the class consisting of phenyl, naphthyl, indolyl, thiophenyl, pyridyl, pyrimidinyl, furyl, pyrrolyl, oxazolyl, thiazolyl, pyrazolyl, isoxazolyl, imidazolyl, isothiazolyl, oxadiazolyl, triazolyl, thiadiazolyl, pyridazinyl, pyrazinyl, triazinyl, indolyzinyl, isoindolyl, benzo[b]furanyl, benzo[b]thiophenyl, indazolyl, benzthiazolyl, benzimidazolyl, quinolinyl, isoquinolinyl, purinyl, quinolizinyl, cinnolinyl, pthalaninyl, quinoxalinyl, napthyridinyl, pteridinyl and quinazolinyl, wherein said aryl or heteroaryl moiety is optionally substituted with one or more moieties selected from the class consisting of the halogen atoms, straight or branched alkyl of 1 to 6 carbons, and —OR¹²⁰ (wherein R¹²⁰ is hydrogen or alkyl of 1 to 6 carbon atoms),
(b) a heterocyclic group selected from the class consisting of imidazolinyl, imidazolidinyl, pyrrolinyl, pyrrolidinyl, piperidinyl, piperazinyl, morpholinyl, thiomorpholinyl, thiazolidinyl, azepinyl, tetrahydropyranyl, tetrahydrofuranyl, benzodioxolyl, tetrahydrothiophenyl and sulfolanyl, wherein said heterocyclyl is optionally substituted with one or more halogen, straight or branched alkyl of 1 to 6 carbons, or —OR¹²¹ (wherein R¹²¹ is hydrogen or alkyl of 1 to 6 carbon atoms), or
(c) straight or branched alkyl of 1 to 7 atoms, wherein said alkyl moiety is optionally substituted with one or more moieties selected from the class consisting of the halogen atoms, straight or branched alkyl of 1 to 6 carbons, and —OR¹²² (wherein R¹²² is hydrogen or alkyl of 1 to 6 carbon atoms),
(ix) —CHO,
(x) the halogen atoms, and
(xi) aryl or heteroaryl which is selected from the class consisting of phenyl, naphthyl, indolyl, thiophenyl, pyridyl, pyrimidinyl, furyl, pyrrolyl, oxazolyl, thiazolyl, pyrazolyl, isoxazolyl, imidazolyl, isothiazolyl, oxadiazolyl, triazolyl, thiadiazolyl, pyridazinyl, pyrazinyl, triazinyl, indolyzinyl, isoindolyl, benzo[b]furanyl, benzo[b]thiophenyl, indazolyl, benzthiazolyl, benzimidazolyl, quinolinyl, isoquinolinyl, purinyl, quinolizinyl, cinnolinyl, pthalaninyl, quinoxalinyl, napthyridinyl, pteridinyl and quinazolinyl,
(J) the halogen atoms, and
(K) —CN and,
wherein R^{1a} is R¹⁰⁰;
E is —N(R¹)—, —N(H)—, —N(SO₂R¹)—, —N(S(O)R¹)— or —N(C(O)R¹)—, where R¹ is as defined above;
G is —O—, —S— or —N(H)—;
X is an oxygen or sulfur atom;
R³ is:
(A) a hydrogen atom, or
(B) branched or unbranched alkyl of 1 to 3 carbon atoms or cycloalkyl of 3 to 5 carbon atoms wherein said alkyl or cycloalkyl group is optionally substituted with:
(i) a group of the formula —OR⁴⁸, wherein R⁴⁸ is a hydrogen atom, or an alkyl or acyl group of 1 to 7 carbon atoms, or
(ii) a group of the formula —NR⁴⁹R⁵⁰, wherein R⁴⁹ and R⁵⁰ are each, independently, a hydrogen atom, alkyl of 1 to 2 carbon atoms, or acyl of 1 to 2 carbon atoms;
R⁴ is a group of the formula —(CR⁵¹R⁵²)ₓ(CR⁵³R⁵⁴)_{y}R⁵⁵, wherein,
x is 0 or 1,
y is 0 or 1,
R⁵¹, R⁵² and R⁵³ are each, independently:
(A) a hydrogen atom,
(B) a group of the formula —R⁵⁶, wherein R⁵⁶ is a hydrogen atom, or an alkyl or acyl group of 1 to 7 carbon atoms, or
(C) branched or unbranched alkyl of 1 to 3 carbon atoms or cycloalkyl of 3 to 5 carbon atoms,
R⁵⁴ is:
(A) a group of the formula R⁵⁷, wherein R⁵⁷ is independently selected from the same class as is R¹, or
(B) a group of the formula —OR⁵⁸, wherein R⁵⁸ is independently selected from the same class as is R¹;
R⁵⁵ is:
aryl or heteroaryl which is selected from the class consisting of phenyl, naphthyl, indolyl, thiophenyl, pyridyl, pyrimidinyl, furyl, pyrrolyl, oxazolyl, thiazolyl, pyrazolyl, isoxazolyl, imidazolyl, isothiazolyl, oxadiazolyl, triazolyl, thiadiazolyl, pyridazinyl, pyrazinyl, triazinyl, indolyzinyl, isoindolyl, benzo[b]furanyl, benzo[b]thiophenyl, indazolyl, benzthiazolyl, benzimidazolyl, quinolinyl, isoquinolinyl, purinyl, quinolizinyl, cinnolinyl, pthalaninyl, quinoxalinyl, napthyridinyl, pteridinyl and quinazolinyl, wherein one or more of the hydrogen atoms of said aryl or heteroaryl group is optionally and independently replaced with:
(A) R⁵⁹, which is aryl or heteroaryl selected from the class consisting of phenyl, naphthyl, indolyl, thiophenyl, pyridyl, pyrimidinyl, furyl, pyrrolyl, oxazolyl, thiazolyl, pyrazolyl, isoxazolyl, imidazolyl, isothiazolyl, oxadiazolyl, triazolyl, thiadiazolyl, pyridazinyl, pyrazinyl, triazinyl, indolyzinyl, isoindolyl, benzo[b]furanyl, benzo[b]thiophenyl, indazolyl, benzthiazolyl, benzimidazolyl, quinolinyl, isoquinolinyl, purinyl, quinolizinyl, cinnolinyl, pthalaninyl, quinoxalinyl, napthyridinyl, pteridinyl and quinazolinyl, wherein one or more of the hydrogen atoms of said aryl or heteroaryl group is optionally and independently replaced with:
(i) branched or unbranched alkyl of 1 to 6 carbon atoms or cycloalkyl of 3 to 6 carbon atoms, which alkyl or cycloakyl group is optionally mono- or polysubstituted with halogen or oxo,
(ii) a group of the formula —COOR⁶⁰, wherein R⁶⁰ is straight or branched alkyl of 1 to 5 carbon atoms or cycloalkyl of 3 to 5 carbon atoms,
(iii) a group of the formula —NR⁶¹R⁶², wherein R⁶¹ and R⁶² are each, independently, a hydrogen atom, alkyl or fluoroalkyl of 1 to 6 carbon atoms, cycloalkyl of 3 to 6 carbon atoms or acyl of 1 to 7 carbon atoms, or wherein R⁶¹ and R⁶² constitute a saturated hydrocarbon bridge of 3 to 5 carbon atoms which together with the nitrogen atom between them form a heterocyclic ring,
(iv) a group of the formula —CONR⁶³R⁶⁴, wherein R⁶³ and R⁶⁴ are each, independently, a hydrogen atom, alkyl or fluoroalkyl of 1 to 6 carbon atoms or cycloalkyl of 3 to 6 carbon atoms, or wherein R⁶³ and R⁶⁴ constitute a saturated hydrocarbon bridge of 3 to 5 carbon atoms which together with the nitrogen atom between them form a heterocyclic ring,
(v) a group of the formula —OR⁶⁵, wherein R⁶⁵ is a hydrogen atom, or an alkyl, fluoroalkyl or acyl group of 1 to 7 carbon atoms,
(vi) a group of the formula —SR⁶⁶, wherein R⁶⁶ is a hydrogen atom, or an alkyl, fluoroalkyl or acyl group of 1 to 7 carbon atoms,
(vii) —CN,
(viii) nitro, or
(ix) halogen,
(B) methyl, which is optionally mono- or polysubstituted with fluorine atoms and additionally is optionally monosubstituted with R⁵⁹,
(C) branched or unbranched alkyl of 2 to 6 carbon atoms or cycloalkyl of 3 to 6 carbon atoms, which alkyl or cycloakyl group is optionally mono- or polysubstituted with halogen or oxo,
(D) a group of the formula —COOR⁶⁷, wherein R⁶⁷ is straight or branched alkyl of 1 to 5 carbon atoms or cycloalkyl of 3 to 5 carbon atoms,
(E) a group of the formula —NR⁶⁸R⁶⁹, wherein R⁶⁸ and R⁶⁹ are each, independently, a hydrogen atom, alkyl or fluoroalkyl of 1 to 6 carbon atoms, cycloalkyl of 3 to 6 carbon atoms or acyl of 1 to 7 carbon atoms, or wherein R⁶⁸ and R⁶⁹ constitute a saturated hydrocarbon bridge of 3 to 5 carbon atoms which together with the nitrogen atom between them form a heterocyclic ring, and wherein one of R⁶⁸ and R⁶⁹ may additionally be the group R⁵⁹,
(F) a group of the formula —CONR⁷⁰R⁷¹, wherein R⁷⁰ and R⁷¹ are each, independently, a hydrogen atom, alkyl or fluoroalkyl of 1 to 6 carbon atoms or cycloalkyl of 3 to 6 carbon atoms, or wherein R⁷⁰ and R⁷¹ constitute a saturated hydrocarbon bridge of 3 to 5 carbon atoms which together with the nitrogen atom between them form a heterocyclic ring, and wherein one of R⁷⁰ and R⁷¹ may additionally be the group R⁵⁹,
(G) a group of the formula —COR⁷², wherein R⁷² is a hydrogen atom, straight or branched alkyl of 1 to 5 carbon atoms, cycloalkyl of 3 to 5 carbon atoms or R⁵⁹,
(H) a group of the formula —OR⁷³, wherein R⁷³ is a hydrogen atom, an alkyl, fluoroalkyl or acyl group of 1 to 7 carbon atoms, or R⁵⁹,
(I) a group of the formula —SR⁷⁴, wherein R⁷⁴ is a hydrogen atom, an alkyl, fluoroalkyl or acyl group of 1 to 7 carbon atoms, or R⁵⁹,
(J) —CN,
(K) nitro, or
(L) halogen;
R⁵ is Cl or trifluoromethyl;
Z is ═N— or ═C(R⁶)— wherein R⁶ is a hydrogen, fluorine, chlorine, bromine or iodine atom, methyl or trifluoromethyl; and,
R⁷ is a hydrogen, fluorine, chlorine, bromine or iodine atom, methyl, —CN, nitro or trifluoromethyl, with the condition that when Z is ═N— or ═C(H)—, R⁷ is chlorine, trifluoromethyl, —CN or nitro;
or a pharmaceutically acceptable salt thereof.

2. A method for the treatment or prophylaxis of inflammatory or immune cell-mediated diseases which comprises administering to a host in need or such treatment or prophylaxis a therapeutic or prophylactic amount of a compound in accordance with claim 1.

3. The method of claim 2 wherein the disease or condition is selected from the group consisting of adult respiratory distress syndrome, shock, oxygen toxicity, multiple organ injury syndrome secondary to septicemia, multiple organ injury syndrome secondary to trauma, reperfusion injury of tissue due to cardiopulmonary bypass, myocardial infarction or use with thrombolysis agents, acute glomerulonephritis, vasculitis, reactive arthritis, dermatosis with acute inflammatory components, stroke, thermal injury, hemodialysis, leukapheresis, ulcerative colitis, necrotizing enterocolitis and granulocyte transfusion associated syndrome.

4. The method of claim 2 wherein the disease or condition is selected from the group consisting of psoriasis, organ/tissue transplant rejection, graft vs. host reactions and autoimmune diseases including Raynaud's syndrome, autoimmune thyroiditis, dermatitis, multiple sclerosis, rheumatoid arthritis, insulin-dependent diabetes mellitus, uveitis, inflammatory bowel disease including Crohn's disease and ulcerative colitis; and systemic lupus erythematosus.

5. The method of claim 2 wherein the disease or condition is asthma.

6. The method of claim 2 wherein the condition is toxicity associated with cytokine therapy.

7. The method of claim 2 wherein the disease or condition is psoriasis.

8. A pharmaceutical composition comprising a compound in accordance with claim 1.
